(19) United States
(12) Patent Application Publication
(10) Pub. No.: US 2005/0130157 A1
(43) Pub. Date: Jun. 16, 2005

(54) Electrophoretic ratchets and cyclic electrophoresis

(76) Inventors: Philip Serwer, San Antonio, TX (US); Gary A. Griess, San Antonio, TX (US); Jonathan W. Valvano, Austin, TX (US); Arnab Basu, Santa Clara, CA (US)

Correspondence Address: FULBRIGHT & JAWORSKI L.L.P., 600 CONGRESS AVE., SUITE 2400, AUSTIN, TX, 78701, US

(21) Appl. No.: 10/502,250

(22) PCT Filed: Jan. 22, 2003
(86) PCT No.: PCT/US03/02007

Related U.S. Application Data
(60) Provisional application No. 60/351,079, filed on Jan. 22, 2002.

(51) Int. Cl. C12Q 1/68

(52) U.S. Cl. 435/6; 204/547; 435/7.1

(57) ABSTRACT

Systems and methods for improving the resolution of macromolecules during electrophoresis. A method of creating an electrical field-rectifying fractionation-ratchet includes obtaining a fractionated particle that has an electrophoretic mobility that varies when an electric field varies, applying a pulsed electrical field to the fractionated particle, and varying a plurality of pulses of the electrical field repeatedly. Another method includes preparative gel electrophoresis that utilizes a continuous fractionation method made possible by an electrophoretic ratchet-generating field. Another method includes implementing cyclic electrophoresis including analyzing a sample by constant field electrophoresis; and enhancing the sample by an electrophoretic ratchet. Another method includes error checking during cyclic electrophoresis comprising: analyzing a sample by constant field electrophoresis; checking for errors in the sample; and enhancing the sample by an electrophoretic ratchet.

This application claims priority to, and incorporates by reference, U.S. Provisional Application Ser. No. 60/351,079, which was filed on Jan. 22, 2002.

### STATEMENT AS TO RIGHTS TO INVENTIONS MADE UNDER FEDERALLY-SPONSORED RESEARCH AND DEVELOPMENT

Aspects of this invention were made with United States Government support under contract to the National Institutes of Health (HG02233 and GM24365). The Government may therefore have certain rights in this invention.

### BACKGROUND OF THE INVENTION

1. Field of the Invention

The invention relates generally to the field of electrophoresis. More particularly, the invention relates to electrophoresis of macromolecules. Specifically, a preferred implementation of the invention relates to improving the resolution of macromolecules during electrophoresis.

2. Discussion of the Related Art

Electrophoresis refers to the migration of charged electrical species when dissolved, or suspended, in an electrolyte through which an electric current is passed. Cations migrate toward the negatively charged electrode (cathode) and anions are attracted toward the positively charged electrode (anode). Neutral solutes are not attracted to either electrode. Conventionally, electrophoresis has been performed on layers of gel or paper.

Because amino acids and proteins are charged nucleic acids, they migrate in an electric field at appropriate pH values. In the most common form of electrophoresis, the sample is applied to a stabilizing medium which serves as a matrix for the buffer in which the sample molecules travel.

A molecular ratchet is a device for rectification of the molecular (nanometer-scaled) effects of either an external field or thermal motion. A nanometer-scaled version of a conventional ratchet and pawl has been used for demonstration of the molecular level consequences of the second law of thermodynamics. One consequence is that rectification of thermal motion is possible only if either a temperature gradient exists or an external source of energy is used. In biological systems (isothermal), an external source of energy (ATP, for example) must be used for rectification of thermal motion. “Thermal ratchet” is the name given to a device that produces rectification of thermal motion. Driving biological motors with a thermal ratchet is an idea embedded in pre-molecular biology literature. This type of idea has been more explicitly proposed for the contraction of muscle, the packaging of bacteriophage DNA, motion along microtubles, processive motion of RNA polymerase along a DNA molecule, and the secretion of protein molecules. A ratchet that is part of a macromolecular motor may be called a motor-ratchet.

Pulsed electrical fields were originally used during gel electrophoresis to increase the resolution of double-stranded DNA molecules much longer than the single-stranded DNA molecules used for DNA sequencing ladders. These procedures of PFGE (Pulsed field gel electrophoresis) were based on matching the pulse durations to the relaxation times of either the fractionated DNA molecules or the supporting matrix. This type of PFGE may be called relaxation-based PFGE. The results of relaxation-based PFGE with DNA sequencing ladders have generally not been good enough to justify adoption of relaxation-based PFGE for current methods of large-scale DNA sequencing. These earlier procedures of PFGE did not involve increasing the length of the effective path of electrophoresis. They also did not involve error-flagging.

A current limitation of large-scale DNA sequencing is the speed and accuracy of assembling and closing sequences. Reading of longer nucleotide sequences is needed to simplify both assembling-closing of sequences in general and error-free assembly of repeated sequences in particular. A second limitation is that errors sometimes occur during the reading of nucleotide sequences. The primary cause of these errors is compression of the resolution of some types of DNA sequencing fragments, often G-C-rich. Errors in nucleotide sequence can thwart establishing of accurate genotype-phenotype correlation. Establishing this correlation is a major objective of genomics.

The resolution of capillary electrophoresis is the factor that limits the readable length of DNA sequencing ladders during capillary DNA sequencing. Numerous attempts have been made to improve this resolution. However, past efforts have the following limitations: (1) they usually requires laboratory reagents-techniques-expertise that are not easy to transfer to other laboratories; (2) no systematic strategy is being used for progressively improving the resolution. Conventional technologies do not circumvent fundamental physical effects that cause reduced peak spacings and increased peak widths; and (3) none of the current work addresses the problem of errors in reading nucleotide sequence.

The gel electrophoretic fractionation of nucleic acids has also been plagued by the following problem: as the length of a molecule increases, eventually DNA length-dependent band separation is lost. During gel electrophoresis, the separation of bands formed by the longest DNA molecules is compressed.

Shortcomings mentioned above are not intended to be exhaustive. Rather, they show that although conventional techniques have demonstrated at least a degree of utility, room for significant improvement remains. In particular, it would be advantageous to have improved electrophoresis techniques.

### SUMMARY OF THE INVENTION

There is a need for the following embodiments. However, the invention is not limited to these embodiments.

According to an aspect of the invention, a method of creating an electrical field-rectifying fractionation-ratchet includes obtaining a fractionated particle that has an electrophoretic mobility that varies when an electric field varies, applying a pulsed electrical field to the fractionated particle, and varying a plurality of pulses of the electrical field repeatedly. According to another aspect of the invention, a method includes preparative gel electrophoresis that utilizes a continuous fractionation method made possible by an electrophoretic ratchet-generating field

According to another aspect of the invention, a method of implementing cyclic electrophoresis includes analyzing a sample by constant field electrophoresis and enhancing the sample by an electrophoretic ratchet. A method of error checking during cyclic electrophoresis includes analyzing a sample by constant field electrophoresis, checking for errors in the sample, and enhancing the sample by an electrophoretic ratchet if a probability of an error is detected. A method of creating a finctional field-rectifying fractionation-ratchet, including obtaining a fractionated particle that has an electrophoretic mobility that varies when a field varies; applying a pulsed field to the fractionated particle; and varying a plurality of pulses of the field repeatedly.

According to another aspect of the invention, a composition for a separation matrix for use in electrophoresis includes a polyethylene-oxide (PEO) and a buffer solution.

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The drawings accompanying and forming part of this specification are included to depict certain aspects of the invention. A clearer conception of the invention, and of the components and operation of systems provided with the invention, will become more readily apparent by referring to the exemplary, and therefore nonlimiting, embodiments illustrated in the drawings, wherein like reference numerals (if they occur in more than one view) designate the same elements. The invention may be better understood by reference to one or more of these drawings in combination with the description presented herein. It should be noted that the features illustrated in the drawings are not necessarily drawn to scale.

FIG. 1 illustrates cyclic electrophoresis in a capillary. The sample is a four-color DNA sequencing ladder. The following are shown: (a) the middle of the first analysis-stage, (b) the end of the first analysis-stage, (c) the first enhancement-stage, (d) the second analysis-stage. The “D” indicates a detector. The “O” indicates origins. An arrowhead indicates DNA sequencing fragments that have left a capillary after being analyzed. The arrow indicates the direction of electrophoresis during an analysis-stage.

FIG. 2 illustrates ratchet-based inverted fractionation for an enhancement-stage. (a) During application of E_{H}, a DNA ladder (four bands=i-iv) is poorly resolved as it migrates away from the origin (O). (b) The ladder is resolved when E_{L} is applied in a direction opposite to the direction of E_{H}. When viewed from the origin, the longest DNA (iv) has migrated further than the shortest DNA (i). (c) The two pulses are adjusted so that the shortest DNA is near the origin.

FIG. 3 illustrates use of software for programming a window of ZIFE (zero-integrated field electrophoresis). The various finctions are described in the text.

FIG. 4 illustrates programming a ZIFE with embedded pulses. A high frequency sawtooth is superimposed on E_{H}. Pulses for FIGE (field inversion gel electrophoresis) replace E_{L}.

FIG. 5a-5d illustrate a reptation-based fractionation-ratchet for DNA molecules. (a) Electrical field pulses for long-pulse ZIFE are shown. (b) An illustration is shown of band-forming DNA fragments during the DNA length-resolution of a resolving E_{L}-pulse (lane ii) that follows a non resolving E_{H}-pulse (lane i). (c) The profile of DNA bands is shown for a two-dimensional gel electrophoresis in which a constant E was used in the first dimension (arrow I) and long-pulse ZIFE was used in the second dimension (arrow II). (d) A strategy for continuous preparative fractionation is demonstrated. A barrier-gel is placed at both the E_{L}-and the E_{H}-ends of the gel used for fractionation during long-pulse ZIFE.

FIG. 6a-6b illustrate a trapping-based fractionation-ratchet for DNA-protein complexes. (a) The profile of bands is shown for T7 DNA-capsid complexes that migrate in the E_{L}-direction (C-DNA in the figure) while band-forming capsid-free DNA molecules (DNA in the figure) migrate in the E_{H}-direction, during long-pulse ZIFE. (b) The trapping of a single DNA-capsid complex is shown.

FIG. 7a-7b illustrate single-particle fluorescence microscopy of the trapping of ethidium-stained T7 DNA-capsid complexes. (a) A spatial field of DNA-capsid complexes is shown while the electrical field is zero. (b) The spatial field in (a) is shown after introducing an electrical field of 4 V/cm. The direction of the electrostatic force on the DNA molecules is indicated by an arrow.

FIG. 8a-8e illustrate the effect of removing an electrical field that has trapped spheres. Latex spheres 240 nm in radius (Polysciences, Warington, Pa.) were trapped in a 0.2% agarose gel by use of E=3 V/cm. The buffer was 0.025 M sodium phosphate, pH 7.4, 0.001 M MgCl₂, 0.1% Triton X-100. The direction of the electrostatic force during trapping is indicated by an arrow. (a) Phase contrast light microscopy was used to image a spatial field of trapped spheres. (b)-(d) Phase contrast light microscopy was used to image the same spatial field after reducing the electrical field to zero. The times after reducing the electrical field to 0 were 0.2, 0.4, and 0.8 seconds, respectively. Differences in the appearance of spheres are caused by differences in focus. A line indicates a fixed point in space for the best-focused spheres. (e) The average values of ΔX (field-parallel) and ΔY (field-perpendicular) are plotted as a function of time both during and after trapping. An electrical field of 3 V/cm was applied at the time indicated by 3 V/cm in the figure. This electrical field was reduced to 0 at the time indicated by 0 V/cm in the figure. The average value of both ΔX (solid line) and ΔY (dashed line) is plotted as a finction of time. The average included a minimum of 150 measurements on randomly selected particles.

FIG. 9 illustrates a ratchet-based procedure for continuous preparative fractionation with multiple fractions. Portions of a single sample are repeatedly loaded in a sample well in the middle of a circular gel (the sample well is labeled S in the figure). After the first portion is loaded, ratchet-based electrophoresis is performed by serially repeating two pulses: E_{H}, for a time, t_{H}, and, then, E_{L} for a time, t_{L}, in directions indicated in the figure. If E_{H}.t_{H}=E_{L}.t_{L}, then a particle will migrate vertically, if μ_{H}=μ_{L}(i.e., ø=0). A particle will migrate between the μ_{L}-direction and vertical, if the magnitude of μ_{H} is less than the magnitude of μ_{L}. This condition sometimes occurs because of either complete or incomplete trapping. The particle will migrate in the μ_{L}-direction in the case of complete trapping in a time short compared with the times of pulses. A particle will migrate in a direction between vertical and the E_{H}-direction if the magnitude of μ increases as the magnitude of E increases. This condition occurs when DNA molecules reptate. Thus, the direction of net migration (dashed line) is determined by μ_{H}/μ_{L}. Particles of each type from all portions loaded (the portions were loaded at different times) accumulate at a unique location in the barrier gel, assuming that both the temperature and E values have been accurately reproduced throughout the fractionation.

### DESCRIPTION OF ILLUSTRATIVE EMBODIMENTS

The invention and its various features and advantages are explained more fully with reference to the nonlimiting embodiments that are illustrated in the accompanying drawings and detailed in the following description. It should be understood that the detailed description and the specific examples, while indicating preferred embodiments of the invention, are given by way of illustration only and not by way of limitation. Various substitutions, modifications, additions and/or rearrangements within the spirit and/or scope of the underlying inventive concept will become apparent to those skilled in the art from this detailed description.

The context of the invention can include increased length resolution of macromolecules, such as polymers, through the electrophoretic process.

The invention can be included in a kit. The kit can include some, or all, of the components that compose the invention. The kit can be an in-the-field retrofit kit to improve existing systems that are capable of incorporating the invention. The kit can include software, firmware and/or hardware for carrying out the invention. The kit can also contain instructions for practicing the invention. Unless otherwise specified, the components, software, firmware, hardware and/or instructions of the kit can be the same as those used in the invention.

The terms a or an, as used herein, are defined as one or more than one. The term another, as used herein, is defined as at least a second or more. The terms including and/or having, as used herein, are defined as comprising (i.e., open language). The term approximately, as used herein, is defined as at least close to a given value (e.g., preferably within 10% of, more preferably within 1% of, and most preferably within 0.1% of). The term substantially, as used herein, is defined as at least approaching a given state (e.g., preferably within 10% of, more preferably within 1% of, and most preferably within 0.1% of). The term program or phrase computer program, as used herein, is defined as a sequence of instructions designed for execution on a computer system. A program, or computer program, may include a subroutine, a function, a procedure, an object method, an object implementation, an executable application, an applet, a servlet, a source code, an object code, a shared library/dynamic load library and/or other sequence of instructions designed for execution on a computer system. The phrase “enhancing the sample”, as used herein, is defined as increasing the resolution of the electrophoretic bands of the sample that is being electrophoresed.

Electrophoretic ratchets may be used for both analytical and preparative electrophoresis. Ratchets, according to embodiments of this disclosure, use a new type of pulsed field. The quality of fractionations using techniques described herein meets the usual standards for biochemistry-based electrophoresis. Supporting media suitable for the techniques here include, but are not limited to, an agarose gel or a capillary-contained polymer solution. The electrophoretic ratchets of this disclosure are effective with a particle that has an electrophoretic mobility (μ=velocity/electrical field) that varies as the electrical field varies. Such a ratchet developed for DNA molecules is effective, in part, because μ increases in magnitude as the electrical field increases in magnitude. Ratchets developed for both DNA-protein complexes and spheres are effective because of the opposite dependence of μ on electrical field. According to different embodiments, ratchet-based gel electrophoresis can be performed in a continuous, preparative mode.

The ratchet-based capillary electrophoresis of this disclosure provides a necessary component for cyclic capillary electrophoresis, which is a procedure for analyzing a DNA profile in several segments which are separated by electrophoretic enhancements of the DNA profile. Cyclic capillary electrophoresis may be used for increasing both the length and the accuracy of the analysis of a DNA sequencing ladder.

Theoretical studies of thermal ratchets usually discuss the use of ratchets for fractionating macromolecules. This type of ratchet may be called a fractionation-ratchet. Thermal fractionation-ratchets have, apparently, not yet produced results that meet the current biochemical standards for finction. However, electrical field (E)-rectifying fractionation-ratchets have met these standards, and exemplify the particular embodiment of the invention discussed here; alternate embodiments of the invention may use fields other than electrical fields. Even though these functional, electrical field-rectifying fractionation-ratchets do not rectify thermal motion, some of them do depend on thermal motion.

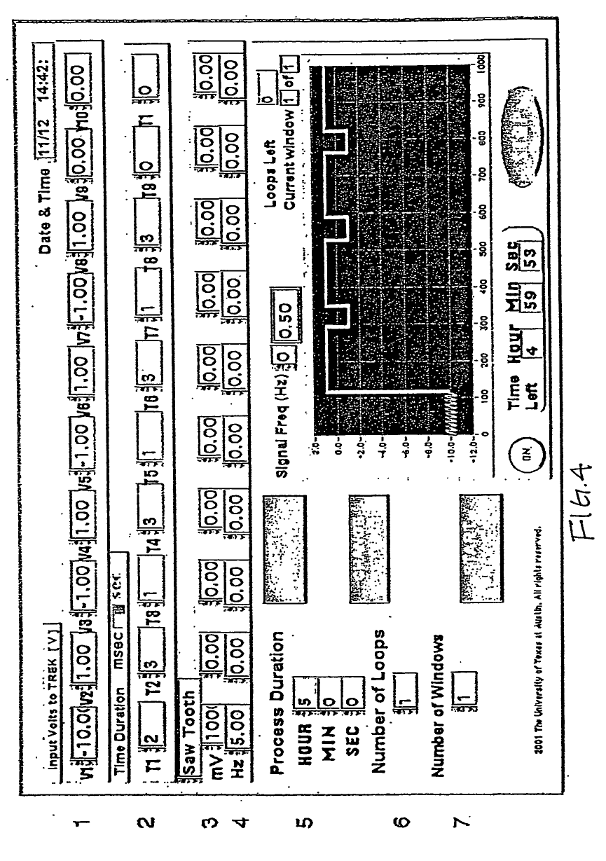

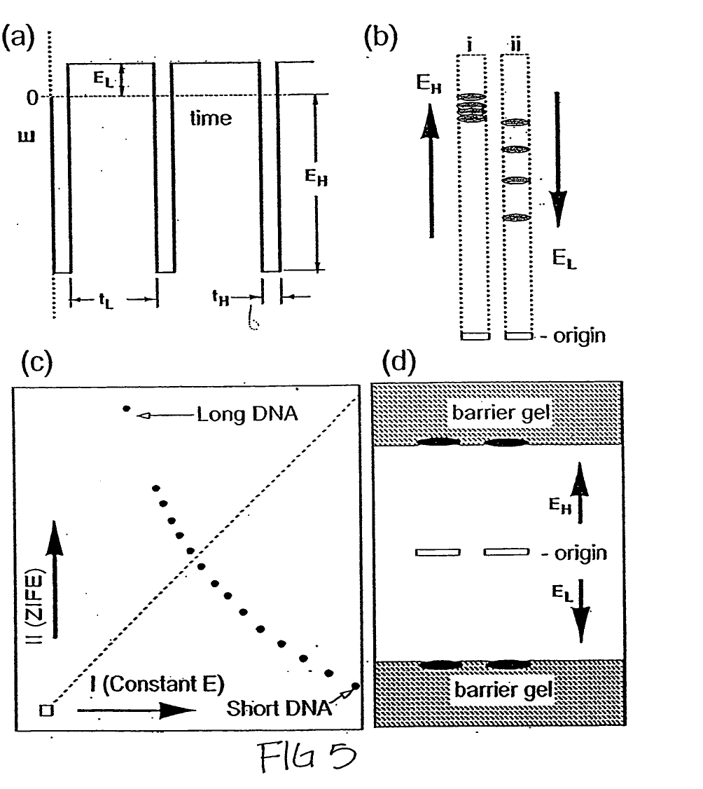

The electrical field used for an electrical field-rectifying ratchet includes fields that include pulses that reverse polarity and change magnitude. The simplest type of ratchet-producing pulsed field starts with a square pulse comparatively high in magnitude (E_{H}) that lasts for a time, t_{H}. This high field pulse is followed by a pulse of both lower magnitude (E_{L}) and longer time, t_{L}. These two pulses may be serially repeated (FIG. 5a). When the time integral of E is zero, the procedure may be called zero-integrated field electrophoresis, or ZIFE (E_{H}.t_{H}=E_{L}.t_{L}).

To have the ratchet work most simply, the following assumption must be accurate: the particles being fractionated have an instantaneous response to changes in E. If pulse times are long in comparison to relaxation times of either the fractionated particle or the fractionating matrix, then this assumption is accurate. In this case, the procedure is called long-pulse ZIFE. Relaxation effects have been exploited during previous studies performed by use of shorter pulses during ZIFE.

Use of long-pulse ZIFE to produce a fractionation-ratchet involves a fractionated particle having an electrophoretic mobility (μ=electrophoretic velocity/E) that varies when E varies. Otherwise, all particles will remain at the origin after long-pulse ZIFE. In the case of square wave ZIFE pulses, μ_{H} is the μ at E_{H}; μ_{L} is μ at E_{L}. In contrast, the concept of μ was originally introduced because most particles have a μ independent of E, in most conditions. The means by which μ is made E-dependent are discussed in subsequent sections. The presence of an E-dependent μ means, in most cases, that a particle will undergo net motion during long-pulse ZIFE. This net motion is the result of rectification (i.e., ratchet-like behavior), because the time integral of E is zero.

One procedure of relaxation-based PFGE is performed by keeping the magnitude of E constant while periodically reversing the direction of E. The net motion is in the forward direction because the time of the forward-directed E is longer than the time of the reverse-directed E. This procedure of PFGE is called field inversion gel electrophoresis, or FIGE. The FIGE technique is popular because FIGE is comparatively inexpensive and does not require great technical expertise.

The resolution of FIGE can be made surprisingly high by (1) making the forward pulse time only slightly different from the reverse pulse time, and (2) tuning the pulse time very carefully. Less than 0.5% difference in DNA length can be resolved for double-stranded DNA molecules approximately 40 kilobase pairs in length.

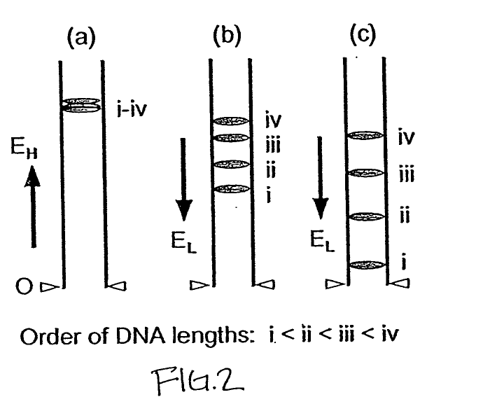

The μ of DNA molecules is E-dependent, based on direct measurement of μ during gel electrophoresis. Without being bound by theory, the magnitude of μ is believed to increase as the magnitude of E increases because DNA molecules undergo E-dependent elongation and end-first migration, a process called biased reptation. Biased reptation causes a progressive loss of DNA length-resolution as both the length of a DNA molecule and the magnitude of E increase. Furthermore, the longer a DNA molecule is, the greater the effect of E on μ is. This latter phenomenon explains the loss of DNA length resolution as the magnitude of E increases. These observations, along with insights from the inventors, lead to the following results concerning long-pulse ZIFE. (a) All DNA molecules will undergo net migration in the E_{H}-direction; (b) The longer a DNA is, the further will be its net migration in the E_{H}-direction. In other words, the DNA fractionation will be inverted in comparison to a conventional constant field fractionation (to be called an inverted fractionation). The reason for inverted fractionation is illustrated by assuming that almost all DNA-length resolution is lost at E_{H}. In this case, most resolution occurs during the E_{L}-pulse in the reverse direction, as shown in FIGS. 2b, 2c, and 5b.

Empirically, inverted fractionation of double-stranded DNA molecules has been shown to occur. This has been shown most directly by use of a two-dimensional agarose gel electrophoresis: (a) a constant field is used in the first dimension, and (b) long-pulse ZIFE is used in the second dimension. FIG. 5c illustrates the result of this two-dimensional electrophoresis: distance migrated in the constant field dimension decreases as DNA length increases. This is the typical fractionation. Distance migrated in the long-pulse ZIFE dimension decreases as DNA length decreases. This is an inverted fractionation. Again, this inverted fractionation is predicted, at least in part, based on the E-dependence of the DNA length-dependence of μ. Empirically, the quality of the DNA bands formed is even better than that of bands formed during constant field electrophoresis.

In the case of some DNA-protein complexes, the magnitude of μ decreases as the magnitude of E increases, in contrast to the pattern for protein-free DNA molecules. Without being bound by theory, the presumed reason is that reptating DNA can enter pores that are smaller than the combined radius of both the DNA and its bound protein. The DNA-protein complex is trapped (μ=0) in these pores if the electrical field is strong enough to prevent reverse thermal motion out of a pore. Without the bound protein, the DNA molecule would not be trapped. Without the bound DNA molecule, much higher fields would be needed to trap the protein. The protein might be either a single molecule or a multimolecular complex.

The following is a bacteriophage T7-derived double-stranded DNA-protein complex that has been electrophoretically trapped in a gel: the 39,937 base pair T7 DNA genome that has bound the T7 protein capsid; the capsid is 60.2 nm in diameter. Infective bacteriophage T7 particles consist of the T7 genome packaged in a cavity of the capsid. To form a T7 DNA-capsid complex, a DNA molecule is expelled from its capsid. Some (but not all) expelled DNA molecules remain bound to their capsid. An electrical field as small as 3 V/cm is sufficient to trap the T7 DNA-capsid complex in an agarose gel.

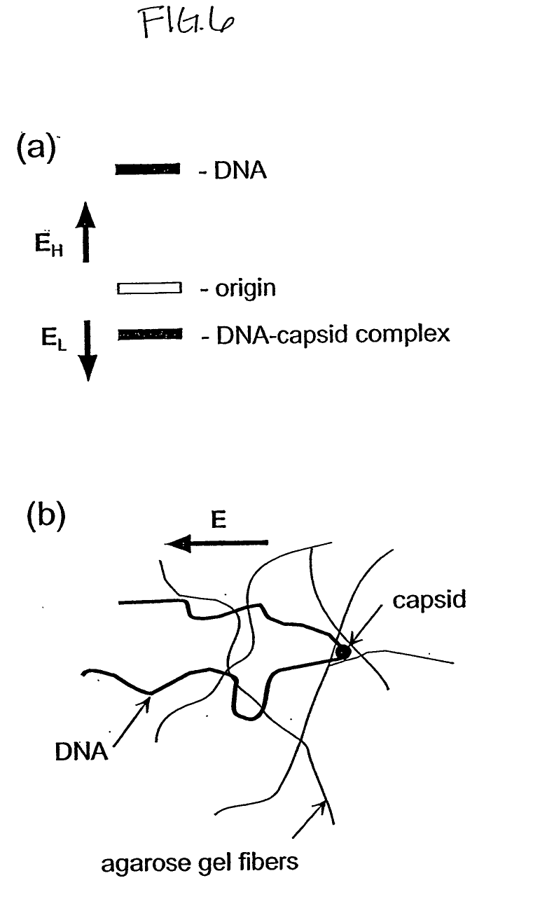

As illustrated in FIG. 6, a long-pulse ZIFE-based ratchet causes the T7 DNA-capsid complex to undergo net motion in the E_{L}-direction because the DNA-capsid complex is trapped in the E_{H}-direction. The long-pulse ZIFE causes the capsid-free T7 DNA molecule to undergo net motion in the E_{H}-direction, for reasons discussed above. Bands are not broadened. The separation between DNA and DNA-capsid complex is large by current standards. Molecular modeling has mimicked the trapping of a DNA-protein complex.

Trapping of DNA-capsid complexes may be observable by fluorescence microscopy of single DNA-capsid complexes, at a resolution of about 200 nm. The T7 DNA genome is 13,580 nm long when pulled straight. Previously, single ethidium-stained DNA molecules have been observed by fluorescence microscopy during gel electrophoresis. To observe the trapping of single T7 DNA-capsid complexes, T7 DNA-capsid complexes were prepared for fluorescence microscopy by embedding in an 0.4% agarose gel that was contained in a miniature apparatus for horizontal electrophoresis. The DNA-capsid complexes were observed both before and during application of an electrical potential (4 V/cm).

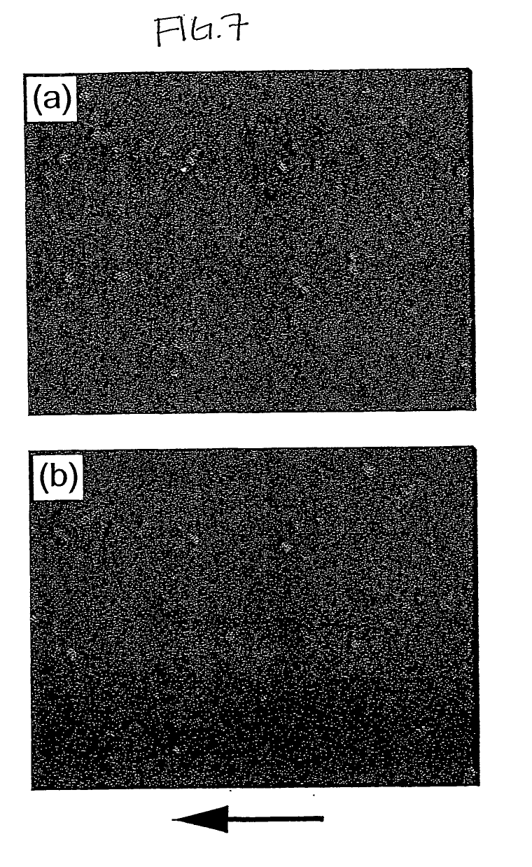

The DNA-capsid complexes appeared to have a random coil-like conformation when not exposed to an electrical field (FIG. 7a). The DNA molecules stretched and migrated when exposed to the electrical field. Most capsid-bound DNA molecules eventually became trapped (FIG. 7a; FIG. 7b). When these molecules became trapped, the stretching increased. The site of trapping was anywhere along a DNA molecule. This observation is in agreement with electron microscopy that reveals the position of the capsid with a resolution (limited by the size of the capsid) of about 60 nm. The DNA-capsid complexes were near the surface of the gel where they appeared to trap more rapidly than they did further inside of the gel. These appear to be the first single-particle images of this type of trapping. These images also illustrate the feasibility of single-particle fluorescence microscopy for observing the dynamics of single DNA-protein complexes in general. Single protein-stained T7 DNA-free capsids have recently been observed in solution by fluorescence microscopy.

Most spheres fractionated by gel electrophoresis are not trapped in most conditions of gel electrophoresis. However, three changes in electrophoretic conditions favor trapping in the case of spheres: (a) increasing the magnitude of E, (b) increasing the radius, R, of the spheres and, (c) increasing the heterogeneity of pore size of the gel. Trapping is experimentally detected either by measuring μ(E) or by performing long-pulse ZIFE. Alternatively, trapping is observed directly by light microscopy during gel electrophoresis. In the case of latex spheres with R≧200 nm, phase contrast light microscopy may be used to observe trapping. These comparatively large spheres are usually completely trapped. That is to say, neither electrophoretic nor thermal motion occurs.

In analogy with the ZIFE-based fractionation-ratchet effective with E_{H}-trapped DNA-protein complexes, a similar fractionation-ratchet is effective in causing E_{H}-trapped spheres to undergo net migration in the E_{L}-direction during long-pulse ZIFE. Biasing the ratchet in the E_{H}-direction causes the untrapped spheres to migrate in the E_{H}-direction, while the trapped spheres migrate in the E_{L}-direction. Thus, the direction of electrophoresis can be a measure of the radius (R) of a sphere because trapping increases as R increases. This type of fractionation has been successful with spheres (bacteriophages particles, latex spheres) as small as 30 nm in radius. One apparent factor for success with the smaller spheres is the use of a gel with a distribution of pore sizes that is broader than usual. This broader distribution of pore sizes may increase the number of pores small enough to act as traps. Spheres migrate through the larger pores and are trapped in the smaller pores. The μ of trapped spheres at E_{H} is reduced in magnitude. However, μ_{H} is not zero in the case of trapped spheres with R between 30 nm and 50 nm. Thus, these smaller spheres are not irreversibly trapped by E_{H}. E values were as high as 30 V/cm.

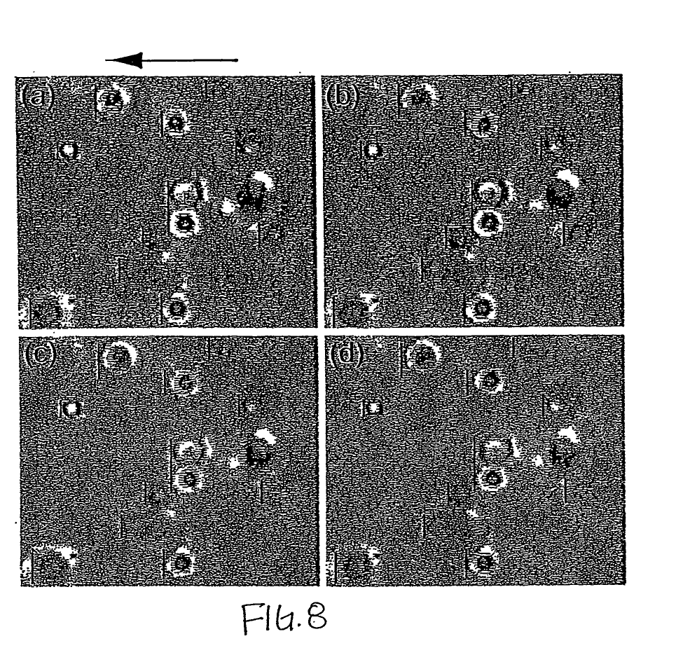

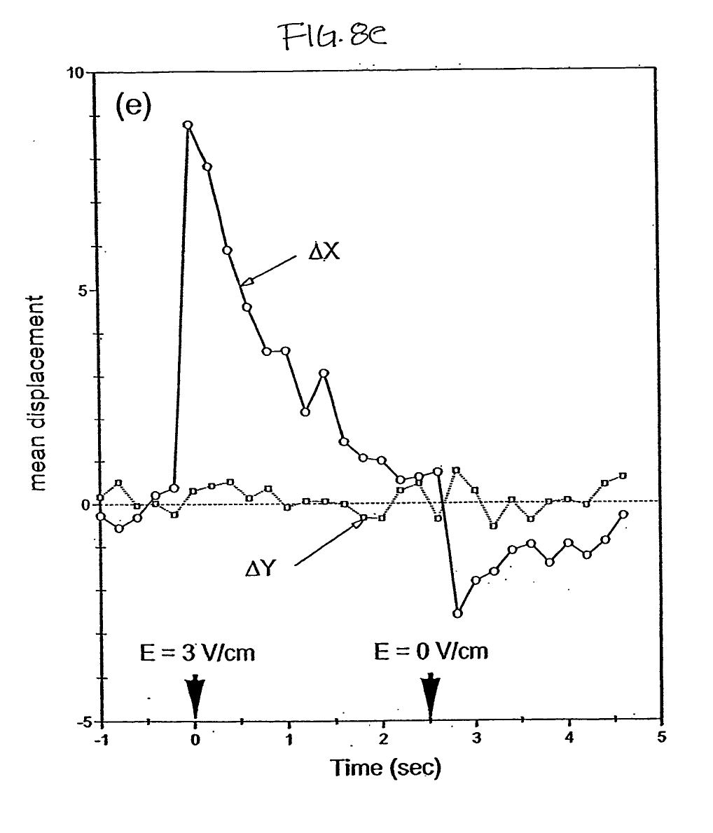

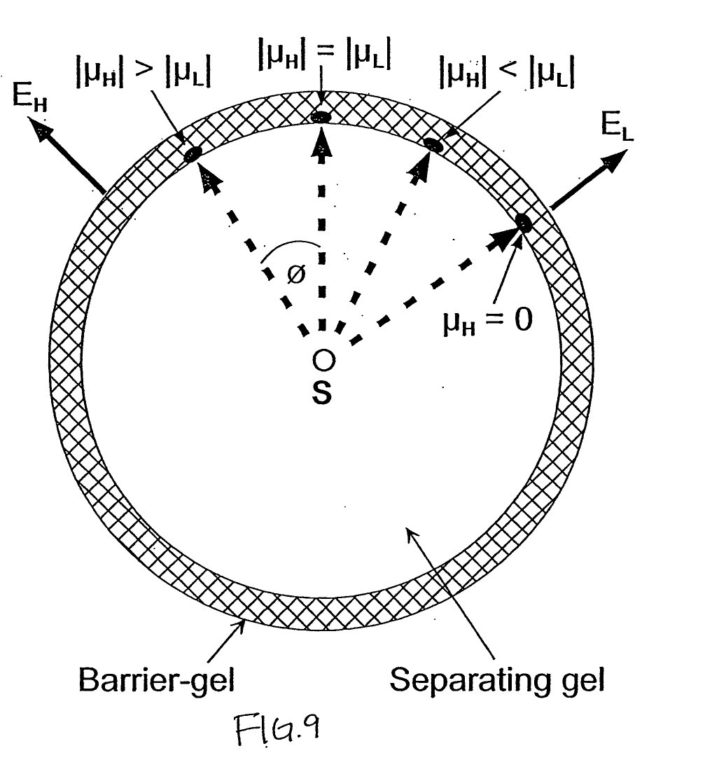

In contrast, trapped spheres 200 nm in radius are irreversibly trapped on this time scale, even when E is only 3 V/cm. The process of trapping includes motion close to the agarose fibers that form the gel. Analysis of fiber-proximal motion is an important component in understanding of the sieving of gels. Trapped spheres, when the trapping field is suddenly changed from 3 V/cm to 0 V/cm, recoil in a direction opposite to the direction of the electrophoretic motion that was occurring just before the spheres were trapped. This phenomenon is shown in FIG. 8. The spheres are trapped in FIG. 8a. The trapping field is made 0, after FIG. 8a. The recoiling spheres are shown in FIG. 8b-8d. For each of several spheres, a line has been drawn to indicate a point fixed in space among the four frames (FIG. 8a-8d). The recoil resembles the second half of a collision with the fibers that originally trapped the spheres.

To obtain a quantitative, statistical analysis of the recoil in FIG. 8b-8d, the following is plotted in FIG. 8e. (a) The latex spheres' average electrical field-parallel displacement (ΔX) in a time interval of 0.2 seconds is averaged and plotted on the vertical axis. (b) The time after the time of reduction of the field to zero (time=0) is plotted on the horizontal axis. The start of this plot is marked by “E=0” and encompasses the positive values of the time in FIG. 8e. After reduction of the field to 0, the averaged values of ΔX immediately became negative, i.e., motion occurred in a direction opposite to the direction of the electrophoretic force that had trapped the spheres. The average ΔX value initially increased in magnitude as the time increased and particles recoiled (FIG. 8e). Then, the average ΔX value decreased in magnitude to a value close to 0. In contrast, average displacement (ΔY) in the field-perpendicular direction remained constant and close to zero throughout the period of recoil (dashed line in FIG. 8e).

The recoil in FIG. 8e occurred because of previous trapping at 3 V/cm. The previous trapping is quantified via the ΔX values for the negative times in FIG. 8e. The time of introducing the electrical field is indicated by E=3 V/cm in FIG. 8e. The average ΔX value was positive, i.e., motion occurred in the direction of the electrophoretic force. The average magnitude progressively decreased as time increased and particles became trapped. At least 150 displacements were used for each time in FIG. 8e. The initial trapping and the subsequent recoil split a sphere-gel fiber collision into two segments.

Gel electrophoresis may not only be used for analytical fractionation, but may also be used for preparative fractionation. Preparative fractionation is more efficient in a continuous fractionation mode. In this mode, a sample is repeatedly loaded at a unique location in a gel; each fraction is continuously collected at another, unique location. An electrophoretic ratchet may be used to make continuous fractionation possible. The simplest example is an extension of the concept of FIG. 5b. This extension is to place a barrier at both ends of a lane used to fractionate a sample. This barrier can be a gel (barrier-gel) with a pore size low enough so that the sample cannot enter the barrier-gel. The sample may be repeatedly loaded in the same sample well. The sample may be continuously fractionated with a ratchet-generating pulsed field. Particles continuously accumulate in two fractions: One fraction is at the barrier in the E_{H}-direction and the other fraction is at the barrier in the E_{L}-direction (illustrated in FIG. 5d). This type of fractionation has been demonstrated in practice for intact bacteriophage particles.

The preparative fractionation of FIG. 5d produces only two fractions. However, a continuum of fractions is possible. One non-limiting means for producing a continuum of fractions is to change the angle separating the E_{H} and E_{L} pulses. This angle is π radians in long-pulse ZIFE. To produce a continuum of fractions, (a) the angle separating the E_{H} and E_{L} pulses may be made less than π radians, while (b) the magnitudes of these two pulses are kept unequal. The smaller angle can be achieved through totally electronic means. Alternatively, this angle can be achieved through rotation of a circular gel. In this latter case, the sample may be continuously loaded in the center of the gel. The angle (ø in FIG. 9) of the direction of migration is uniquely determined by the ratio, μ_{H}/μ_{L}. Thus, particles continuously accumulate in a circular barrier-gel at a position determined by this ratio. If μ_{H}=μ_{L}, then all particles will accumulate at ø=0, so that no fractionation will occur.

Improved resolution should also be possible during electrophoresis if an analysis can be stopped and then repeated after an intervening electrophoresis that enhances the fractionation. To achieve this type of intervening electrophoresis, the direction of electrophoresis may be reversed by use of a procedure that increases the separation between peaks. If the direction of a constant field electrophoresis is reversed without changing the magnitude of E, then the separation between peaks (or bands, depending on whether a gel or capillary method is used) decreases. Thus, a constant E cannot be used for electrophoresis in the reverse direction. But, if an electrophoretic ratchet is used in the reverse direction, the separation between peaks can increase. In order to increase this separation during electrophoresis in the reverse direction, the fractionation must be inverted.

The DNA fractionation-ratchet described above produces an inverted fractionation. Thus, use of this ratchet in the reverse direction results in reverse migration that increases the separation between peaks (bands) that was achieved in a previous forward, constant field electrophoresis. Several forward-reverse cycles can be used. The net effect is to produce a virtual origin of electrophoresis that is outside of the capillary. That is to say, the effective length of the electrophoretic path may be increased, even though the physical length of the capillary is not increased. This increase in effective length is a source of increase in resolution. The profile of peaks may be read during any or all of the stages of constant field electrophoresis.

In theory, no limit exists to the resolution that can be achieved by cyclic electrophoresis. Thus, resolution-limited procedures such as, but not limited to, capillary DNA sequencing may benefit. In practice, the resolution-enhancing capacity of cyclic electrophoresis has been demonstrated both for agarose gel electrophoresis and for capillary electrophoresis in a solution of ungelled polymer. The major limitations are the following: (a) thus far, the total time of electrophoresis must be increased to achieve the increase in resolution; (b) analysis of the profile of bands (peaks) must be performed in several segments, and additional effort-software is needed to assemble the segments; and (c) flexibility is limited by the requirement to read each segment before the molecules involved migrate out of the separating medium. During ratchet-based cyclic capillary electrophoresis of DNA molecules, broadening of peaks causes surprisingly small, if any, loss of resolution.

A strategy is described here for increasing both the resolution and the flexibility of capillary electrophoresis performed in a sieving medium of ungelled polymer. This strategy is based on analysis that is done in several stages of constant field electrophoresis. Enhancement-stages are between the analysis-stages. An enhancement-stage (1) increases the separation between peaks, while (2) moving DNA molecules slightly in the reverse direction. An enhancement-stage may be based on an electrophoretic ratchet generated by a pulsed electrical field that can be zero-integrated. The ratchet-generating pulses may typically be longer than the field pulses that have previously been used to improve the resolution of DNA molecules. No limit has been found to the resolution enhancement achievable. Apparently, diffusion-induced peak broadening is inhibited and, in some cases, may be reversed by the ratchet. The enhancement-stages are dependent on the electrical field-dependence of a plot of electrophoretic mobility as a function of DNA length. To generate the pulsed electrical field, a computer-controlled system with a time resolution of 30 microseconds has been developed, although other systems with different time resolutions are contemplated by the techniques described herein. Programming is flexible enough to embed other pulses within ratchet-generating pulses. These other pulses may be, for example, either shorter field-inversion pulses or high frequency periodic oscillations previously found to sharpen peaks.

Another sieving medium or separation matrix that may be used is polyethylene-oxide (PEO). The use of PEO as a separation matrix for DNA sequencing was tested under several conditions, including: total PEO concentration, buffer concentration, electric field strength, and capillary temperature. The conditions are summarized as follows.

To determine the concentration of PEO to be used in electrophoresis, PEO of three molecular weights were used: high (H) 8,000,000; medium (M) 2,000,000; and low (L) 600,000. Solutions were made by slowly dissolving polymer in buffer while stirring with a magnetic bar. Stirring was continued for at least 24 hours. As a rule, for a given concentration (w/vol), the higher molecular weight PEO was more viscous and more difficult to dissolve. Different combinations of H, M and L PEO were investigated. Best results were obtained with a 3% total PEO concentration consisting of 2% H and 1% L. This seems to be near the limit of both solubility and viscosity for filling capillaries on the ABI 310 genetic analyzer. Standard 50 μm ID 47 cm capillaries from ABI were used without any conditioning. (Because of the design of the ABI-310 instrument, cleaning or conditioning of the capillary is not practicable.) Capillaries were used for over 100 runs without significant deterioration of results.

In one embodiment when using PEO, the buffers in the electrophoresis reservoirs and the polymer solutions may be made from the same stock (Sigma B-4930 10×Capillary Electrophoresis Running Buffer) Dilutions ranged from 1× to 0.01×. Electropherograms were obtained over the entire range of buffer concentrations even though the instrument reported zero current for the more dilute buffers. In the more dilute buffers the sample fluorescence signal was relatively diminished. This was compensated for by increasing the sample concentration, injection voltage and injection time.

The applied electric field may be programmed in kilovolts (kV) on the ABI-310 instrument. The field strength in V/cm may be obtained by dividing by the effective capillary length (50 cm). The range of fields tested was 2.5 to 15 kV. Because the maximum voltage output of the Trek 10/10B power amplifier is 10 kV an intermediate value of 7.5 kV was most frequently chosen for comparison with pulsed modes.

The ABI-310 may be designed to hold the capillary temperature between 5° and 60° above ambient. The maximum on the control panel is 75° C. Runs were made at 40°, 50°, 60° and 70° C. for different combinations of buffer and electric field for the 3% PEO mixture described above.

To prepare electrophoretic samples, ROX-2500 size standard markers labeled with red fluorescent Big Dye (ROX) were obtained from ABI (Warrington, UK). Sequence standard ladders were prepared by PCR labeling of puc119 cloning vector using the Big Dye labeling kit. Single-strand DNA was obtained by denaturation in formamide at 90° C. then quenched on ice.

The ABI-310 records raw data in proprietary files as four parallel color channels collected at a fixed rate. These data files can be analyzed by both the ABI sequencing software and the ABI Gene-scan software. For quantitative purposes, the Gene-Scan software is more useful because it can handle 2× longer data files, and it has automatic peak detection, tabulating in exportable file format the position, height and area of each peak. These three values are used in a spreadsheet program to calculate resolution. Resolution is defined as the ratio of two terms: the selectivity (peak separation per nucleotide) and peak width. To visualize how resolution depends on these two terms, it is useful to plot them separately as a function of DNA length (L, nucleotides). The selectivity and peak width are expressed in the same units which, for convenience, are kV.min. Thus, the resolution is a dimensionless value which is a function of L. The selectivity is obtained from the derivative of the plot of peak position vs. L. This plot can be fitted by a third-order polynomial, so the selectivity is a second-order polynomial of L. The peak width is proportional to the peak area divided by the peak height. Experimentally, the peak width varies with L as a second-order polynomial. Sequencing requires that the resolution be equal to or greater than unity, so the maximum read length can be found by the solution of the two polynomial functions at the condition of equality.

Use of the 3% PEO solution described above achieved separations with resolution comparable to the commercial POP-6 polymer preparation from Applied Biosystems (Foster City, Calif.). The PEO solutions had the following advantages: a) they do not contain urea denaturant which is known to break down, and b) the buffer concentration could be varied at will. The PEO solution's low ionic strength may also contribute to sharper peaks and minimizing peak compression in electrophoresis. Although the lower concentrations of PEO did not have as good an overall resolution, there was relatively higher separation of the high moleculear weight DNA.

Apart from the exceptions noted below, variation of buffer strength, electric field strength and capillary temperature had only slight effects on resolving power. That is, the selectivity and peak width plots were pretty much superimposable within the limits of variation. Occasionally, a trial would yield plots quite different from the norm, but repetition under the same conditions failed to reproduce this difference. If the cause of these anomalies were known, it could lead toward optimization of resolution. Results were poorest at the extremes of the variable parameters, i.e. 40° and 70° C.; 0.01× and 1× buffer strengths. A dependence on electric field strength at long DNA length was observed for selectivity but not for peak width. This indicates that ZIFE can be used with PEO solutions as well as POP-6.

Capillary electrophoresis of DNA molecules may be used for the analysis of genotypes. Both double- and single-stranded DNA molecules may be used. Improved resolution of DNA molecules benefits several applications of capillary electrophoresis in the analysis of genotypes. In the case of double-stranded DNA molecules, exemplary applications include (a) mutation analysis via the length polymorphism of restriction endonuclease-digested DNA, (b) analysis of the number of short tandem repeats, and (c) construction of hybrid-free DNA libraries. In the case of single-stranded DNA molecules, exemplary applications include both increasing the readable length of DNA sequencing ladders and decreasing the error frequency of DNA sequencing.

Cyclic electrophoresis has increased the separation of DNA peaks when used with ungelled polymer in a capillary, thereby increasing resolution. The data also suggest that cyclic capillary electrophoresis can reduce, and possibly reverse, peak broadening, also improving resolution. Resolution may be continuously increased until something other than resolution becomes the major limitation. In the case of detection by the fluorescence of labeled DNA molecules, the intensity of fluorescence may become a limitation for the longer DNA molecules in a DNA ladder.

In addition to progressive increase in resolution, cyclic capillary electrophoresis may be used to analyze the same collection of DNA molecules more than once. This latter capability may help in reducing errors in the interpretation of DNA profiles. For example, G-C rich DNA sequencing fragments are “error prone” during DNA sequencing. A primary source of errors is a single-stranded DNA secondary structure that sometimes causes G-C rich DNA fragments to be in the wrong position, when the assumption is made that μ decreases smoothly in magnitude as the length of a DNA molecule increases. To probe for this type of error by cyclic capillary electrophoresis, a first constant field analysis-stage may be followed by a second constant field analysis-stage of the same DNA sequencing fragments. The second analysis-stage may be separated from the first analysis-stage by an intervening enhancement-stage that probes for errors.

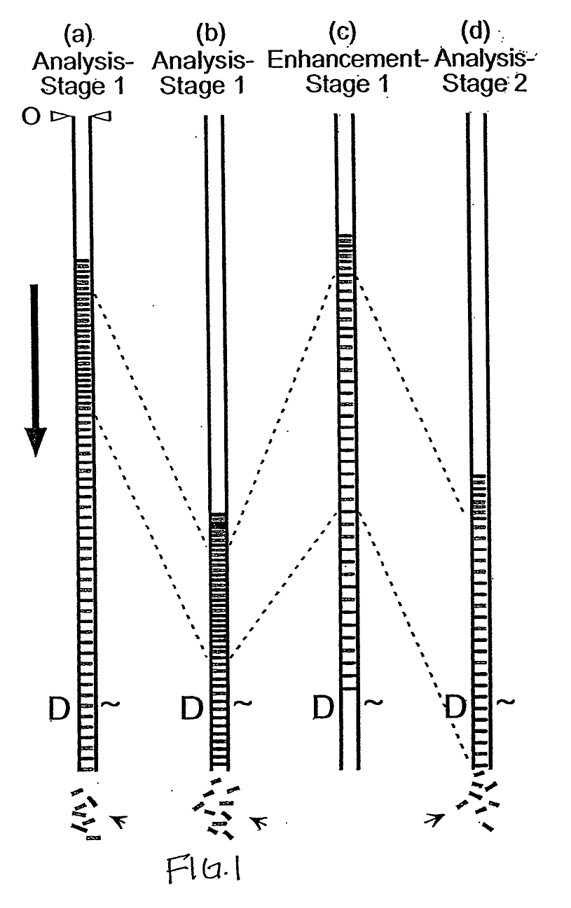

Cyclic capillary electrophoresis of DNA fragments begins with a conventional analysis by constant field electrophoresis (FIG. 1a; the sample is a four-color DNA sequencing ladder).

Peak width increases with time during constant field capillary electrophoresis but is also a finction of both the electrical field and the type-concentration of the sieving matrix at the least. Pulsing can further complicate the understanding of peak widths either through effects on the sieving matrix (shear-induced thinning or entanglement-disruption, for example) or effects on the DNA molecule. The latter effects might include (1) changes in DNA orientation caused by induced DNA-dipole interaction with an oscillating field, and (2) local DNA condensation caused by condensed counterions. The possibility of the latter is suggested by the observation that pulsed fields can alter intermolecular aggregation of double-stranded DNA molecules. Although pulsed fields do not cause intermolecular aggregation of DNA sequencing ladders, intramolecular condensation (i.e., globule formation) is still a possibility. The bottom line is that the quality of DNA peaks during PFCE may need to be determined empirically, if it can not be predicted at present.

During constant field capillary electrophoresis, the most rapidly-migrating (shortest) DNA fragments migrate out of the origin-distal end of the capillary during this first analysis-stage (arrowhead in FIG. 1a). The electrophoresis is stopped when the DNA fragments are not well enough resolved for the purposes of the analysis (FIG. 1b).

Then, an enhancement-stage interrupts the first analysis-stage. The enhancement-stage causes slight reverse migration of the DNA molecules, while it causes the separation between peaks to increase (FIG. 1c). Optimally, the net motion in the reverse direction is as small as possible, to delay reaching the origin-end of the capillary. Because of the reverse migration, care must be taken not to let any of the unanalyzed portion of the ladder migrate out of the original-proximal end of the capillary.

Just after the first enhancement-stage ends, a second cycle begins with the second analysis-stage (FIG. 1d). The second analysis-stage may perform a re-analysis of some of the DNA fragments analyzed by the first analysis-stage. This re-analysis may be useful for increasing accuracy of analysis, as mentioned above. Re-analysis is also useful for assembly of the data from neighboring analysis-stages of the various cycles. Photobleaching-induced reduction of peak height during the first analysis-stage can also be used to assist assembly, if fluorescence is used for detection. Photobleaching reveals the last peak analyzed during the previous analysis-stage.

The analysis-enhancement cycle illustrated in FIG. 1 can be repeated. Both the analysis- and the enhancement-stages can be varied in length. Separation between peaks can be increased indefinitely. Resolution may be proportionately increased if diffusion-induced peak-broadening does not occur. Thus, two significant requirements of cyclic electrophoresis are the following: (a) a procedure must exist for increasing peak-separation while the average DNA molecule undergoes a comparatively small net movement in the reverse direction. That is to say, the peaks must undergo an expanding, “accordion-like” movement; and (b) the procedure for increasing peak-separation must work while either minimizing or, optimally, reversing peak-broadening. Both requirements are met by the techniques of the present disclosure.

From the beginning, both band and peak quality were very promising during long-pulse ZIFE. Double-stranded DNA molecules form unusually sharp bands during inverted fractionation by long-pulse ZIFE in an agarose gel. These bands are sharper than the bands formed during uninterrupted constant field agarose gel electrophoresis. Peaks are also sharp after the use of long-pulse ZIFE-based enhancement-stages, during capillary electrophoresis. These observations are important because most methods known to increase peak spacing during DNA sequencing (e.g., increasing the physical length of the capillary and lowering E) also result in diffusion-induced peak-broadening. The long-pulse ZIFE-based procedures increase peak separations, but do not increase peak widths.

Inverted DNA fractionation is sometimes observed, but only for a limited range of DNA lengths, during constant field gel electrophoresis. However, a complete, systematic inverted DNA fractionation is preferred for the enhancement-stages of cyclic capillary electrophoresis. Thus, the electrical field is varied during an enhancement-stage.

Previous studies have improved the fractionation of DNA molecules by use of an electrical field that is varied in discrete pulses (pulsed field gel electrophoresis, or PFGE). However, fractionations by the conventional PFGE are usually not inverted. These fractionations are based on matching pulse times to a relaxation time of either the DNA molecules, the gel or both. Nonetheless, PFGE both can be and has been used to achieve completely inverted fractionations. The pulses used for inverted fractionation are longer than the previously-used relaxation-based pulses. Thus, these procedures of PFGE are called long-pulse procedures.

To visualize how a long-pulse procedure works, imagine that DNA molecules at the origin of a gel are initially subjected to a pulse that has a comparatively high electrical field, E_{H}. The time of this pulse is t_{H}. The DNA fragments migrate with comparatively poor separation, because of E_{H}-induced reptation (FIG. 2a). For illustration, FIG. 2a has assumed almost no resolution during the E_{H}-pulse. Next, the electrical field is both lowered in magnitude (E_{L}) and inverted. The time of the E_{L}-pulse is t_{L}. The DNA molecules now migrate with much better separation because of the lower electrical field. The net result of the two pulses is that the order of the DNA fragments is inverted, when viewed from the origin (FIG. 2b). This pair of pulses can be repeated, so that the separation between peaks becomes progressively larger. The values of t_{H} and t_{L} in FIG. 2 can be chosen to keep the shortest DNA molecules near the origin after a cycle is complete (FIG. 2c).

Analysis of electrophoretic profiles may be a complex process during cyclic electrophoresis in a slab gel. However, this analysis is much simpler during capillary electrophoresis because the sieving medium is not manipulated between analysis-stages. Cyclic capillary electrophoresis of double-stranded DNA molecules has been used to increase the separation of peaks formed by double-stranded DNA fragments. The sieving medium may be an ungelled solution of polymer. The concept is basically the same for capillary electrophoresis as it is for gel electrophoresis.

To achieve an inverted fractionation, attempts are made to avoid relaxation effects during the enhancement-stages of a cyclic capillary electrophoresis. To determine whether these attempts are successful, t_{H} is varied while keeping the ratio, t_{H}/t_{L}, constant. Relaxation effects are negligible if and only if the fractionation does not significantly vary as t_{H} varies. That is to say, absence of relaxation effects is equivalent to a fractionation that is independent of the scale of pulse times. In practice, fractionation independent of the scale of pulse times is observed for long-pulse ZIFE during the agarose gel electrophoresis of double-stranded DNA molecules. In contrast, significant dependence on the scale of pulse times is observed during capillary electrophoresis of double-stranded DNA molecules in an ungelled solution of hydroxymethyl propyl cellulose. This dependence is found for t_{H} values over 10 seconds when E_{H}=200 V/cm ands E_{L}=200 V/cm.

The resolution of DNA peaks improves (a) as the separations between the peaks increase, and (b) as the peak widths decrease. A long-pulse ZIFE-based enhancement-stage increases the separation between peaks. The same peak can be analyzed both before and after an enhancement-stage of long-pulse ZIFE.

The width of a DNA peak (1,500 base pairs) was measured both before and after 0, 1, 2 and 3 enhancement stages of long-pulse ZIFE. Each enhancement-stage (E_{H}=200 V/cm, E_{L}=20 V/cm in 1.0% hydroxypropyl methyl cellulose) lasted 1.0 hr. The results were 0.44, 0.50, 0.40 and 0.40, respectively. Peak widths were not increasing and appeared to be slightly decreasing.

High frequency periodic oscillations may cause the sharpening of peaks when these oscillations are superimposed on a constant field during the capillary electrophoresis of double-stranded DNA molecules. Also, long-pulse ZIFE has a band-sharpening effect when used during the agarose gel electrophoresis of double-stranded DNA molecules. The cause of the sharpening effects of electrical field oscillations is not known.

The following describes one embodiment of cyclic electrophoresis, as applied to DNA sequencing. Initially, the shortest DNA fragments of a four-color DNA sequencing ladder are analyzed by constant field capillary electrophoresis. This initial part of the procedure is the same as the constant field analysis performed by conventional procedures. This initial analysis constitutes the first analysis-stage of a cyclic capillary electrophoresis. The forward direction is defined to be direction of DNA motion in this analysis-stage, i.e., the direction defined by the DC constant field.

The first analysis-stage continues until the DNA length-resolution is not sufficient for base-calling. Then, this constant field analysis-stage is stopped. Next, the first enhancement-stage is inserted. During this first enhancement-stage, the DNA ladder is driven in the reverse direction by use of a pulsed field of long-pulse ZIFE. The pulsed field produces an inverted fractionation. Thus, the separation between DNA peaks increases, while the shortest unanalyzed DNA molecules move slowly away from the detector. This reverse-directed PFCE is illustrated in FIG. 2c; the detector is indicated by “D”. The peak spacing is increased by increasing the effective length of the capillary. Care is taken so that the long DNA-end of the DNA sequencing ladder does not migrate out of the origin-end of the capillary. Finally, the first enhancement-stage is stopped. This completes the first cycle.

The second cycle starts with a second constant field-based analysis-stage. Typically, this second analysis-stage uses the same E that the first analysis-stage uses although that is not required. However, the analysis begins at a longer DNA sequencing fragment. This DNA sequencing fragment was near, but not in, in the zone of compression during the first analysis-stage. To overlap the first two analysis-stages, this fragment is a fragment that has already been analyzed. The combination of both the first enhancement-stage and the second analysis-stage resolves this DNA sequencing fragment to the extent that it is distant from the zone of compression. Thus, as the second analysis-stage continues, DNA sequencing fragments are analyzed that were in the zone of compression during the first analysis-stage. The second analysis-stage continues until the resolution of the DNA sequencing ladder is not sufficient for base calling. Additional cycles are then added until some limitation other than resolution prevents accurate base calling. This limitation might be either time or the intensity of the fluorescence labeling of the longer DNA sequencing fragments. Ultimately, the sequences from the various analysis-stages are assembled. Overlapping of analysis-stages can be used to assist assembly.

To generate a pulsed field for cyclic electrophoresis, pulses have been programmed by use of the following user-interface: (a) the interface is organized in a series of windows. Each window programs up to 10 pulses; electrical potential and time of each pulse are entered (rows 1 and 2, respectively of the window of FIG. 3); (b) periodic oscillations (if any) are superimposed on any of the 10 pulses. The type of oscillation is selected from a menu (saw tooth, square wave, sine wave); the amplitude and frequency of a periodic oscillation are entered (rows 3 and 4, respectively of the window of FIG. 3); (c) the total time of a window is entered (row 5, left of the window of FIG. 3); the eclipsed time of a window is displayed during electrophoresis (top right of box at the lower right of the window of FIG. 3); (d) subsequent windows are similarly programmed by clicling on a “SET WINDOW” button (middle of row 5 in the window of FIG. 3); the number of loops through these windows is entered (row 6, left of the window of FIG. 3); the total time remaining for the current window is displayed (middle of the bottom of the box at the lower right in the window of FIG. 3); (e) the program is started by clicking on a “START PROCESS” button (middle of row 7 of the window of FIG. 3); and (f) the program can be changed by clicking on a “CHANGE SETTING” button (middle of row 6 in the window of FIG. 3). As the program is entered, the outcome is graphically displayed (lower right of the window of FIG. 3). This display shows the input that is programmed. It is not a measurement. The program displayed in FIG. 3 is a simple long-pulse ZIFE.

The software for generating windows was developed in the object-oriented programming language, LabView (National Instruments, Austin, Tex.). This software will run either in an IBM-compatible or in a Macintosh environment. The digital output is converted to an analogue electrical potential by a digital-to-analogue converter (PCI-6111; National Instruments). The analogue potential (0-10 volts) is used as the input for an electrical potential-amplifier (Trek, Inc., Medina, N.Y.). The amplifier multiplies this input by a fixed factor. This factor is 1,000 for one amplifier (Trek 10/10B) that has been used; it is 2,000 for another (Trek 20/20B). The response time resolution roughly 30 microseconds.

In the current configuration, the computer-controlled power supply can drive electrophoresis only when the native power supply of the electrophoresis apparatus is connected to a resistive shunt. The shunt must be used in order to prevent the apparatus from sensing an error and stopping all functions (including the analysis functions). The software and hardware may, in a different embodiment, integrate the amplifier into the apparatus so that the amplifier is the only power supply for electrophoresis and the shunt is not needed. The Trek amplifiers are grounded. Safety precautions are stricter than they are with a floating power supply.

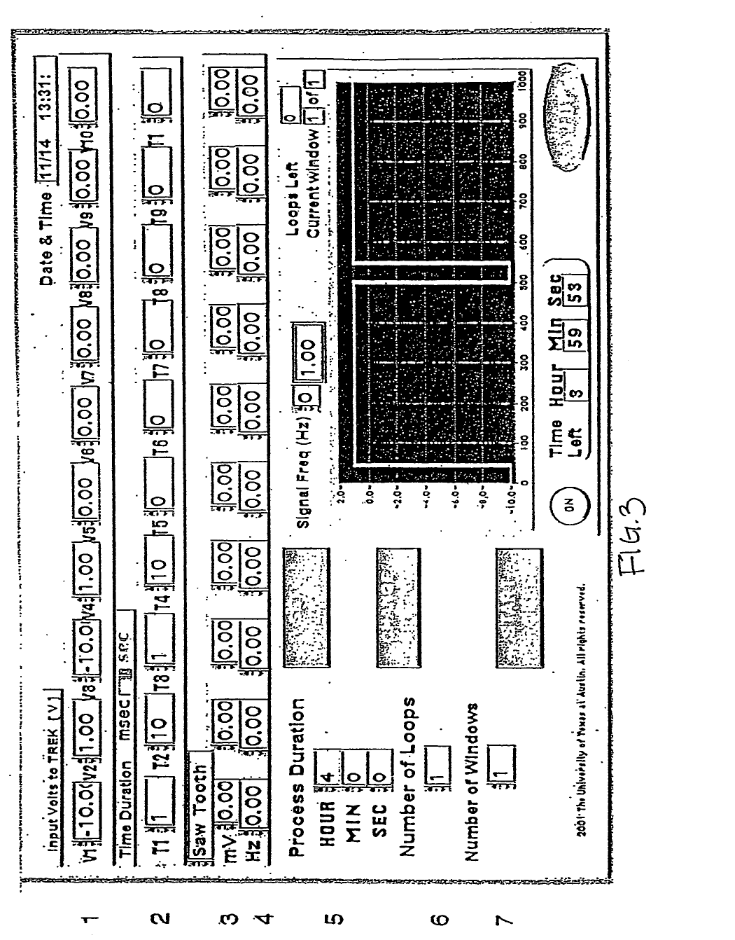

The program of FIG. 3 is a simple long-pulse ZIFE. Fractionations may be improved by embedding the following pulses within the pulses of long-pulse ZIFE: (a) as discussed above, embedding high frequency periodic pulses may sharpen peaks; and (b) embedding previously-developed, relaxation-based trains of pulses may increase the separations between peaks. The following train of pulses is in the latter category: time-asymmetrical pulses produced by change of sign, but not magnitude (called field inversion gel electrophoresis or FIGE). The program of FIG. 4 is a more complex long-pulse ZIFE in which (a) high frequency saw tooth oscillations are superimposed on E_{H}, and (b) FIGE has replaced a constant E_{L}. For a more detailed description of the software embodiment of this invention, please reference Example 1.

Dynamic computer-modeling may be used for predicting the movement of DNA peaks. Such a modeling may be used to estimate the optimal times and fields of the pulses of enhancement-stages. The optimal enhancement-stage may not always be based on a strict long-pulse ZIFE. Biasing pulses in the E_{L}-direction should become progressively more productive as the DNA length increases. The reason is the following: the E_{H}-induced increase in the magnitude of μ becomes greater as DNA length increases. Thus, E_{L}-biasing may be needed to keep the reverse migration from being more rapid than optimal.

Assembly of data from the various analysis-stages must be performed when cyclic electrophoresis is used. This assembly of data is not necessary if the analysis-stages are not separated form each other. Thus, separated analysis-stages introduce both additional effort and chance for assembly errors. Assembly can be achieved by analyzing the same peaks during each of two neighboring analysis-stages, i.e., by overlapping analysis. Photobleaching is not a problem for the second, overlapped analysis-stage, in the case of single-stranded DNA sequencing fragments labeled with the four Bigdyes used for DNA sequencing with an Applied Biosystems machine. In this case, photobleaching causes less than 25% immediate loss of signal during analysis. Most of this loss recovers.

Thus, overlapping analysis is feasible in the case of DNA sequencing. The residual photobleaching may be used in determining the last base analyzed during the previous analysis-stage. Tracking of peaks during the enhancement-stage can assist in this process. Thus, assembly of data can become a fairly complex process, and software may be used to simplify this process, as is shown in the art.

Detection-correction of DNA sequencing errors may become increasingly important as genotype-phenotype correlation becomes a focus of research. In theory, detection of DNA sequencing errors can be enhanced by cyclic capillary DNA sequencing. This is done by re-analysis of peaks after an intervening, error-flagging enhancement-stage is used. However, appropriate pulsing for an error-flagging enhancement-stage should be used. The following shows that appropriate pulsing may be developed: during electrophoresis in a gel, bulge-containing DNA fragments can have a μ that decreases in magnitude as the electrical field increases in magnitude. A fragment can be made to bulge either by binding protein or by partial denaturing, in the case of double-stranded DNA fragment. The reason for error-causing, out-of-position DNA sequencing fragments is, presumably, secondary structure produced by intramolecular base pairing. This secondary structure resembles bulging. Therefore, the field-response of the μ of a DNA sequencing fragment may have enough dependence on secondary structure to flag errors.

The implementation of an error-flagging cyclic capillary electrophoresis may require more than one error-flagging algorithm. These algorithms may be embedded in feedback-activated software that detects possible problems while the sequence is being analyzed for the first time. This is to say, cyclic capillary DNA sequencing may become an exercise in informatics.

Capillary electrophoresis of a DNA sequencing ladder is now routinely used to read nucleotide sequences that are 600-700 bases long. The length of reading can be extended to 1000-1300 by optimizing sample preparation-cleaning, capillary diameter, polymer-based sieving matrix, conditions of electrophoresis (such as the electrical field) and reading software. Reading of even longer nucleotide sequences is prevented by loss of resolution of the longer DNA fragments in a DNA sequencing ladder. This loss of resolution is caused primarily by peak broadening and secondarily by reduced peak spacing.

Increasing the resolution of capillary DNA sequencing may be achieved by increasing the length of the effective electrophoretic path, without increasing the physical length of the capillary. Both detection and correction of errors may be achieved by reading the same DNA sequencing fragments more than once. Thus, the enhancement-stages of cyclic capillary electrophoresis may be used for error-flagging-correcting, as well as increase in resolution. An error-flagging event may be inserted between the two readings of the same DNA sequencing fragments. These concepts require abandoning use of the uninterrupted constant electrical field mode that is currently used for DNA sequencing by capillary electrophoresis. That is to say, a custom-designed, variable (pulsed) electrical field may be used instead of a constant field. Although techniques described herein may apply pulsed fields in capillaries, the results are transferable to microarray electrophoresis.

The inventor's laboratory has improved PFGE to increase the length of the effective path of double-stranded DNA molecules in an agarose gel. This novel version of PFGE is effective for increasing DNA length-resolution. The same path-lengthening concept was successfully used to improve the length-resolution of double-stranded DNA molecules fractionated in an ungelled polymer solution contained in a capillary. The use of a pulsed field with capillary electrophoresis may be called PFCE. More recently, the same path-lengthening concept of PFCE was successfully used to improve the DNA length-resolution of single-stranded DNA molecules in denaturing (sequencing) conditions. The supporting medium was an ungelled, entangled polymer solution contained in a capillary within an Applied Biosystems 310 apparatus for capillary DNA sequencing.

A strategy based on cyclic capillary electrophoresis may transmit improvements via the internet. The software for improved pulsing-base calling can be sent from a data processing center, via the intemet, to every worldwide-user who has compatible equipment for electrophoresis. The additional equipment needed has a cost less than 20% of the cost of the capillary electrophoresis apparatus. Thus, a cyclic capillary electrophoresis-based strategy overcomes the first limitation above. That is to say, cyclic capillary electrophoresis is a paradigrn-shifting procedure that should play a role for DNA sequencing similar to the role that PFGE plays for separating large double-stranded DNA molecules.

A cyclic capillary electrophoresis-based strategy may also provide a platform for overcoming the other limitations. As the database used for cyclic DNA sequencing grows, the new data typically suggests new ways to improve resolution of DNA sequencing fragments. Thus, systematic, progressive improvement in procedures is expected in the case of DNA sequencing by cyclic capillary electrophoresis. Finally, a cyclic, PFCE-based strategy may both detect and correct errors in the reading of the nucleotide sequence.

The E-induced loss of DNA length-resolution is caused by E-induced orientation and stretching of a DNA molecule. After orientation, an elongated DNA molecule migrates end-first during electrophoresis through the fibrous network of a gel. This process is called biased reptation because of the analogy to snake-like movement. Detailed theory has described the process of biased reptation and molecular orientation. Briefly, molecular orientation causes loss of resolution because the resistance to motion becomes roughly proportional to the length of a DNA molecule, if the DNA molecule is oriented by the electrical field. Of course, the electrostatic force is also proportional to DNA length. Thus, the electrophoretic mobility (μ;=velocity/E) becomes independent of DNA length at any given E that has a magnitude high enough to molecular orientation.

The hardware-software for cyclic capillary electrophoresis may be added, without great cost, to any current device for capillary DNA sequencing. Advances in cyclic capillary electrophoresis may be both continuous and internet-communicable. Error detection-correction may be integrated into cyclic capillary DNA sequencing. These are all major advantages.

The particular material selected for the separating medium used in electrophoresis is not essential to the invention, as long as it provides the described function. Particularly in one embodiment, this function may involve behavior that translates into a mobility vs. DNA length plot that varies with the applied field. The slope of the plot may be steeper as the field becomes lower in magnitude in this particular embodiment. One practical limitation in selecting the material is the apparent instability of the sieving characteristics of commercial polymers. Normally, those who make or use the invention will select the best commercially available material based upon the economics of cost and availability, the expected application requirements of the final product, and the demands of the overall manufacturing process.

### EXAMPLES

The following examples are included to facilitate an understanding of ways in which the invention may be practiced. However, it should be appreciated that many changes can be made in the exemplary embodiments which are disclosed while still obtaining like or similar result without departing from the spirit and scope of the invention. Accordingly, the examples should not be construed as limiting the scope of the invention.

### Example 1

A software algorithm and user interface has been created to generate a pulsed field for cyclic electrophoresis, and one embodiment of such a computer program, a software-based virtual lab bench, is described here.

Front Panel

On a front instrumentation panel that is displayed by the software, a user can program a series of ten different voltages (Input Volts) per window using control boxes V1 to V10. This group of voltages may be the actual output from a DAQ (data acquisition) board and is the input of a TREK amplifier. In this embodiment of the software, the maximum magnitude of each voltage is +10 volts and the minimum is −10 volts.

Users may then select the unit of the time between millisecond or second by moving a time unit selector switch (between 1 and 2) to msec or sec, respectively. The default is in milliseconds. Each of the ten Input Volts control boxes, e.g. T1 through T10, contains the time duration of the corresponding Input Volts to the TREK amplifier.

The superimposed signal for electrophoresis is then selected. There are two types of superimposed signals. They are the saw tooth wave (square wave) and the sine wave. The default selection here is the saw tooth wave. The sine wave or saw tooth wave will be added or superimposed on top of the signal of Input Volts to TREK. Each superimposed signal control box corresponds to the control boxes in Input Volts and Time duration. Users can control both magnitude and the frequency of superimposed signal for each control box. Their units are millivolts and Hertz, respectively.

The user may also select time period during which the process will run by entering an amount of time in the appropriate Time duration control box. This sets the time duration of the process. The program will iterate until the time duration has elapsed. If the time is zero and the user attempts to run the program, an error message will be displayed and the process will stop.

By clicking a Set Window button after all the aforementioned desired values are determined,a user will see a pop up window to save the setting of all the values into a datalog file. The default file name is already given in the popup window as window1.dat, window2.dat, and up to window10.dat, for the ten default windows that are provided, but users can choose any file names they like. The user should be careful not to overwrite a previously saved file with the same name. To avoid this problem, it is recommended to create a new directory with a name that represents the experiment, and store all the series of windows in the directory. Later, if the specific set of windows gave a satisfactory result for an experiment, the user can reuse the settings.

After saving a window, a user can go back to that window, make changes in the values, and save the window again by clicking the change setting button. A detailed explanation about the Change Setting panel is in the following section.

When the Change Setting panel is accessed, a series of window settings will iterate from window number 1 to the last window according to the number in the Number of Loops. The user must be aware that this number does not represent an iteration number for a specific window but for all the windows in a series.

The Total Number of Windows control box is useful when a user wants to reuse previous windows and knows the number of total windows that were used. The default value is zero.

The Start Process button is used to start the process after all the values described above have been determined and saved. This will read all of the values from the files in a directory. When the Total Number of Windows is the default value zero, the program will request only the files that are saved through the Set Window button after the program has started. If the number in Total Number of Windows is not zero, then the program will need that number of specified windows. The program will not proceed until it reads all of the windows.

To display the status of output generation from the DAQ board, the On/Off button is depressed. A pause of one hundred milliseconds occurs between windows during execution. This will appear as a flickering of the On/Off indicator.

The Time Left box will display the time that is left to finish the process, and the Date & Time box displays the date and time of the current system while the program is executing. The Loops Left box displays the number of the loops remaining. Zero denotes the last loop. The Total/Current Window displays the total number of windows and the current window number.

The Signal Frequency control box displays the frequencies of the ten input voltages as an array. The left box that has arrows represents the index of the input voltage control box. The right box displays the frequency of the corresponding input voltage. A user can adjust the frequencies by changing the time duration of the input voltages. The displayed frequency changes when the user changes the time duration.

The execution of the program will be aborted whenever the STOP button is pressed. The STOP button will stop the execution of the DAQ board so that there will be no output from the board, This stop button is different from the LabVIEW stop button on the upper left corner. The LabVIEW stop button will not give a stop command to the DAQ board, and the output signal from the board will be continuously generated until the computer power has been turned off or the SequePulse STOP command has been executed.

The Waveform Graph graphically displays the waveform generated by the DAQ board. It also displays an alert, when the instrument safety locks have been triggered and there is a potential high-voltage risk.

Change Setting Panel

The Change Setting panel is used to modify the values that are saved in the datalog files, and will be opened when the Change Setting button on the front panel is pressed. The Change Setting panel can also be opened to modify values in the datalog files via the readwrite.vi.

The number in the Window Number control box specifies the window number that starts from one to ten. The corresponding window will be open to read or write the values.

When the READ button is pressed, a pop up window will show up with the default path and filenames. Choose a file and click on the filename to open. The values in the file will be displayed on the corresponding control boxes. After changes on the necessary values are made, clicking the WRITE button will save the new values on the file. A popup window will show up to make sure the directory and the filename are correct. The new values will be displayed on the front panel as well after the file is saved.

A user can repeat read and write as many times as he or she wants. To finish this panel and to go back to the front panel, click Done button. It will automatically close this Change Setting panel and return to the front panel if this panel was open through the Change Setting button on the front panel. Otherwise it will remain open and stop this VI (virtual instrument).

Control boxes will display the values selected for the voltages, time duration, wave form, and superimposed signal frequency when a file is open, and also can be used as input to change values. The function and meaning of each value have been described.

### Example 2

This Example shows the steps that may be taken to create waveforms from a Seque Pulse front panel similar to the one described in Example 1.

To setup and run the Seque Pulse program:

(1) Open the front panel of Seque Pulse by opening sequepulse4.7.vi file or another desired virtual instrumentation file and run the application.

(2) Enter the necessary control values into the control boxes. In this case, these value may include voltage, time duration, wave form, and superimposed signal frequency, as described earlier in Example 1.

(3) Press the SET WINDOW1 button. A user can see the frequencies of pulses inputted at the Input Volts to TREK in the Signal Freq feature. At this time a user can adjust the frequencies of pulses and superimposed signal. The frequencies will change simultaneously as values in Time Duration are changed. After making sure that all the values are correct, save this window to a file window1.dat by pressing SAVE WINDOW1.

(4) Save the file in a desired location. Make sure that the saving directory or the path is correct. If a new directory is desired, a user may do so and save the file under the directory.

(5) A user can make additional windows by repeating steps 2 to 4, or start the process by clicking START PROCESS. A popup window will open to accept values from the window1.dat file. The process will start after clicking Open.

(6) While the Seque Pulse program is running, the output of the DAQ board is displayed as shown below. If the process has to be aborted at any time, just click the STOP button.
To change waveform sequences:

The Change Setting panel is used to change values in the datalog files which stores the values entered in previous iterations of the Seque Pulse program. This panel can be accessed when the front panel of Seque Pulse is running by clicking the Change Setting button in the front panel. Alternatively, the readwrite.vi file can be opened directly to operate it independently.

To read a file, set the value of window number, as described earlier in Example 1, and then click the READ button. A popup window will show up with a default path or a path that was open previously in the LabVIEW program. Make sure the directory and the path are correct, and then click open.

The values in the *.dat file that is selected are shown in a Change Setting panel. The user may make desired changes in the values and click WRITE to save the values in the file. In this example, the superimposed signal has been changed from saw tooth to sine wave. After all the changes have been saved, click DONE. This panel will be automatically closed and the screen will return to the front panel of Seque Pulse.

The Seque Pulse program can also detect certain errors such as those relating to the electrophoresis equipement. For example, if the access door to the electrophoresis unit is open, the Seque Pulse will automatically stop the process and display a warning message notifying the user of the problem.

### Practical Applications of the Invention

Practical applications of the invention that have value within the technological arts are DNA sequencing, resolution restriction endonuclease analysis, and high resolution VNTR analysis. The latter two analyses are performed with double-stranded DNA. They are used for the rapid typing of pathogenic bacteria, among other organisms. There are virtually innumerable additional uses for the invention, all of which need not be detailed here since they will be apparent to those having skill in the art, with the benefit of this disclosure.

### Advantages of the Invention

Embodiments described in this disclosure have several advantages. (i) The separation (and resolution) of bands (peaks) is progressively improved. without lengthening the separating medium. (ii) The resolution of bands (peaks) may be increased without known limit. The speed of achieving any given level of resolution may be increased by the reduction of band broadening effects of using long-pulse ZIFE. Detection sensitivity may be the practical limit for some DNA ladders. (iii) Anomalous effects (band inversions, for example) may be detectable by comparing results of one stage of electrophoresis with the results of another. Band inversions and possibly other anomalies may be removable by adjusting the pulsing. (iv) Error correction of the analysis may be possible. (v) Continuous preparative fractionation may be possible. (vi) Resolution would also be improved for a ratchet-based fractionation that is not cyclical. Because the μ vs. E relationship for single-stranded DNA has the form of the μ vs. E relationship for double-stranded DNA, these advantages may apply to optimization of the reading of DNA sequencing ladders.

The individual components of this disclosure need not be formed in the disclosed configurations, but could be provided in virtually any configuration.

Further, homologous replacements may be substituted for the substances described herein. Further, agents which are both chemically and physiologically related may be substituted for the agents described herein where the same or similar results would be achieved.

It will be manifest that various substitutions, modifications, additions and/or rearrangements of the features of the invention may be made without deviating from the spirit and/or scope of the underlying inventive concept. It is deemed that the spirit and/or scope of the underlying inventive concept as defined by the appended claims and their equivalents cover all such substitutions, modifications, additions and/or rearrangements.

### REFERENCES

Each of the following references are hereby incorporated by reference in their entirety.

1. U.S. Pat. No. 5,405,519.

2. U.S. Pat. No. 6,147,198.

3. A. Griess , P. Serwer: Biopolymers 29, 1863 (1990).

4. A. Griess, E. Rogers, P Serwer: Electrophoresis 21, 859 (2000).

5. A. Griess, P. Serwer: Electrophoresis, in press (2001).

6. A. Griess, R. A. Harris, P. Serwer: Appl. Theor. Electrophor. 3, 305 (1993).

7. Albarghouthi, M. N. and Barron, A. E. (2000) Polymeric matrices for DNA sequencing by capillary electrophoresis. Electrophoresis 21, 4096-4111.

8. Albarghouthi, M. N., Buchholz, B. A., Doherty, E. A., Bogdan, F. M., Zhou, H. and Barron, A. E.(2001) Impact of polymer hydrophobicity on the properties and performance of DNA sequencing matrices for capillary electrophoresis. Electrophoresis. 22, :737-747.

9. Alberts, R. Miake-Lye: Cell 68, 415 (1992).

10. Bankier, A. T., Meth. Mol. Biol. 2001, 167, 89-100.

11. Biron, M., Berche, P., Ferroni A., Pathologie et Biologie 2001, 49, 128-137.

12. Birren, B. and Lai, E. (1993) Pulsed Field Gel Electrophoresis: A Practical Guide. Academic Press, San Diego, Calif.

13. Birren, B., Lai, E., Pulsed Field Gel Electrophoresis: A Practical Guide, Academic Press, San Diego, Calif., 1993.

14. Birren, E. Lai: Pulsed Field Gel Electrophoresis: A Practical Guide (Academic Press, San Diego, Calif. 1993).

15. Brassard, E., Tunnel, C. and Noolandi, J (1992) Pulsed field sequencing gel electrophoresis. Electrophoresis. 13, 529-535.

16. Brassard, E., Turmel, C., Noolandi, J., Electrophoresis 1992, 13, 529-535.

17. Bruin, G. J. (2000) Recent developments in electrokinetically driven analysis on microfabricated devices. Electrophoresis. 21, 3931-3951.

18. Bustamante, C., Gurrieri, S. and Smith, S. B. (1993) Towards a molecular description of pulsed-field gel electrophoresis. Trends Biotechnol. 11, :23-30.

19. Bustamante, S. Gurrieri, S. B. Smith: Trends Biotechnol. 11, 23 (1993).

20. Bustamante: Annu. Rev. Biophys. Biophys. Chem. 20, 415 (1991).

21. C. Elston: Biophys. J. 79, 2235 (2000).

22. C. Schwartz, M. Koval, Nature 338, 520 (1989).

23. Carle, G. F. (1992) Field-inversion gel electrophoresis. In: Methods in Molecular Biology, Vol. 12, Pulsed-Field Gel Electrophoresis (Burmeister, M. and Ulanovsky, L. Eds.) Humana Press, Totowa, N.J., pp. 3-18.

24. Carrilho, E. (2000) DNA sequencing by capillary array electrophoresis and microfabricated array systems. Electrophoresis. 21, 55-65.

25. Casjens, S., Wyckoff, E., Hayden, M., Sampson, L., Eppler, K., Randall, S. Moreno, E T. and Serwer, P. (1992) Bacteriophage P22 portal protein is part of the gauge that regulates pacling density of intravirion DNA. J. Mol. Biol. 224, 1055-1074.

26. Chakraborty, R., Stivers, D. N., Su, B., Zhong, Y., Budowle, B., Electrophoresis 1999, 20, 1682-1696.

27. Chen, T., and Skiena, S. S. (2000) A case study in genome-level fragment assembly. Bioinformatics 16, 494-500.

28. Chen, X., Baumstark, T., Steger, G. and Riesner, D. (1995) High resolution SSCP by optimization of the temperature by transverse TGGE. Nucleic Acids Res. 23, 4524-4525.

29. Chialvo, D. R., Millonas, M. M., Phys. Lett. A 1995, 209, 26-30.

30. Chu, B., Liu, T., Wu, C. and Liang, D. (2001) DNA capillary electrophoresis using block copolymer as a new separation medium. Methods in Mol. Biol. 162, 225-238.

31. D. Astumian, I. Derenyi: Biophys. J. 77, 993 (1999).

32. D. Keller, C. Bustamante: Biophys. J. 78, 541 (2000).

33. D. R. Chialvo, M. M. Millonas: Phys. Lett. A 209,26 (1995).

34. D. Vale, F. Oosawa: Adv. Biophys. 26, 97 (1990).

35. Dalton, R. (2001) Genetic sleuths rush to identify anthrax strains in mail attacks. Nature 413, 657-658.

36. Demeler, B. (2001) UltraScan ver. 5.0, a comprehensive data analysis software package for analytical ultracentrifugation data. http://www.ultrascan.uthscsa.edu

37. Demeler, B., Behlke, J. and Ristau, O. (2000) Molecular parameters from sedimentation velocity experiments: whole boundary fitting using approximate and numerical solutions of Lamm equation. Meth. Enzymol. 321, 38-66.

38. Desruisseaux, C., Slater G. W. and Kist, T. B. (1998) Trapping electrophoresis and ratchets: a theoretical study for DNA-protein complexes. Biophys. J. 75, 1228-1236.

39. Desruisseaux, C., Slater, G. W., Kist, T. B. L., Biophys. J. 1998, 75, 1228-1236.

40. Desruisseax, G. W. Slater, T. B. Kist: Biophys. J. 75, 1228 (1998).

41. Dolnik, V. (1999) DNA sequencing by capillary electrophoresis. J. Biochem. Biophys. Methods 41, 103-119.

42. Dolnik, V., Gurske, W. A. and Padua, A.(2001) Galactomannans as a sieving matrix in capillary electrophoresis. Electrophoresis. 22,:707-719.

43. Eisenberg, J., Zohar, A., Mei-Tal, G., Steinberg, A., Tartakovsky, E., Gritsenko, I., Nemanov, L., Ebstein, R. P., J. Med. Genet. 2000, 96, 258-261.

44. F. Huxley: Prog. Biophys. Biophys. Chem. 7,255 (1957).

45. Fabret, C., Quentin, Y., Guiseppi, A., Busuttil, J., Haiech, J., Denizot, F., Microbiology 1995, 141, 345-350.

46. G A. Griess, E. Rogers, P. Serwer: Electrophoresis 22, 981 (2001).

47. G. Donnan: Smithsonian Report, 309 (1929).

48. G. W. Slater, P. Mayer, G. Drouin: Methods Enzymol. 270, 272 (1996).

49. G. W. Slater, P. Mayer, S. J. Hubert, G. Drouin: Appl. Theor. Electrophor. 4, 71 (1994).

50. G. Xue., H. M. Pang, E. S. Yeung: J. of Chromatogr. 914, 245 (2001). April 20.

51. Griess, G. A., Rogers, E., Serwer, P., Electrophoresis 2000, 21, 859-864.

52. Griess, G. A., Serwer, P., Electrophoresis, in press.

53. Guldberg, P., Gronbak, K., Aggerhohn, A., Platz, A., Thor Straten, P., Ahrenkiel, V. P. Zeuthen, J., Nucleic Acids Res. 1998, 26 1548-1549.

54. Houdusse, H. L. Sweeney: Curr. Opin. Struct. Biol. 11, 182 (2001).

55. Huang, S. J. Hayes, P. Serwer: J. Struct. Biol., in press (2002).

56. J. Brokaw: Biophys J. 81, 1333 (2001).

57. Keller, C. Bustamante: Biophys. J. 2000, 78, 541-556.

58. Kim, Y. and Morris, M. D., Anal. Chem. 1994, 66, 3081-3085.

59. Kim, Y. and Morris, M. D., Electrophoresis 1996, 17, 152-160.

60. L. Viovy, A D. Defontaines: in Methods Mol. Biol. 12 (Humana Press, Totawa, N.J. 1992), 403.

61. L. Viovy: Rev. Mod. Phys. 72, 813 (2000).

62. Louie, D. and Serwer, P. (1990) Blunt-ended ligation can be used to produce DNA ladders with rung spacing as large as 0.17 Mb. Nucleic Acids Res. 18, 3090.

63. Magnasco, M. O., Phys. Rev. Lett. 1993, 71, 1477-1481.

64. Matsudaira, P., Ehrlich D., Anal. Chem. 2000, 72,3388-3391.

65. Mayer, P., Slater, G. W. and Drouin G. (1994) Simulation of reduced band broadening during single-stranded DNA pulsed field electrophoresis in polyacrylamide gels. Electrophoresis 15,:120-127.

66. Messing, J., Meth. Mol. Biol. 2001, 167, 13-31. 2001.

67. Mitnik, L., Heller, C., Prost, J. and Viovy, J.-L. (1995) Segregation in DNA solutions induced by electric fields. Science 267, 219-222.

68. Morris, M. D., Schwinefus, J. J., de Carmejane, O., Methods Mol. Biol. 2001, 162, 307-321.

69. Noolandi, J. and Turmel, C. (1995) Preparation, manipulation, and pulse strategy for one-dimensional pulsed-field gel electrophoresis (ODPFGE). Mol. Biotechnol. 4, :25-43.

70. Noolandi, R. Forsyth, S. An-Chang: Proc. Natl. Acad. Sci, U. S. A. 90, 10101 (1993).

71. O. Magnasco: Phs. Rev. Lett. 71, 1477 (1993).

72. P. Serwer: Biopolymers 27, 165 (1988).

73. Pang, H. M. and Yeung, E. S. (2000) Automated one-step DNA sequencing based on nanoliter reaction volumes and capillary electrophoresis. Nucleic Acids Res. 28, E73.

74. Pevzner, P. A., Tang, H. and Waterman, M. S. (2001) An Eulerian path approach to DNA fragment assembly. Proc. Natl. Acad. Sci., USA 98, 9748-9753.

75. Pluen, A., Tinland, B., Sturm, J., Weill, G., Electrophoresis 1998 19, 1548-1559.

76. Pohl, H. A. (1978) Dielectrophoresis. Cambridge University Press, Cambridge, Mass.

77. Quesada, M. A.and Menchen, S. (2001) Replaceable polymers for DNA sequencing by capillary electrophoresis. Methods in Mol. Biol. 162, 139-166.

78. R. P. Feynman, R. B. Leighton, M. Sands: The Feynman Lectures on Physics (Addison-Wesley, Reading, Mass.)

79. Richard, G. F., Paques, F., EMBO Reports 2000, 1, 122-126.

80. Richterich, P., Genome Res. 1998, 8, 251-259.

81. Rocha, E. P., Danchin, A. and Viari, A. (1999) Functional and evolutionary roles of long repeats in prokaryotes. Res. Microbiol. 150, 725-733.

82. Rosenblum, B. B., Oaks, F., Menchen, S. and Johnson, B. (1997) Improved single-strand DNA sizing accuracy in capillary electrophoresis. Nucleic Acids Res. 25, 3925-3029.

83. Rousseau, J., Drouin, G. and Slater, G. W. (2000) Gel electrophoretic mobility of single-stranded DNA: The two reptation field-dependent factors. Electrophoresis 21, 1464-1470.

84. Rousseau, J., Drouin, G., Slater, G. W., Electrophoresis 2000, 21, 1464-1470.

85. Rousselet, J., Salome, L., Ajdari, A., Prost, J.,: Nature 1994, 370, 446-448.

86. Rousselet, L. Salome, A. Ajdari, J. Prost: Nature 370, 446 (1994).

87. Sack, G. H., Jr., Adv. Veterinary Medicine 1997, 40, 103-117.

88. Salas-Solano O., Schmalzing, D., Koutny, L., Buonocore, S. Adourian, A., Matsudaira P. and Ehrlich, D. (2000) Optimization of high-performance DNA sequencing on short microfabricated electrophoretic devices. Anal. Chem. 72, 3129-3137.

89. Salas-Solano, D. Schmalzing, L. Koutny, S. Buonocore, A. Adourian, P. Matsudaira, D. Ehrlich: Anal. Chem. 72, 3129 (2000).

90. Salas-Solano, O., Carrilho, E., Kotler, L., Miller, A. W., Goetzinger, W., Sosic, Z., Karger, B. L., Anal. Chem. 1998, 70, 3996-4003.

91. Salas-Solano, O., Schmalzing, D., Koutny, L., Buonocore, S., Adourian, A., Matsudaira, P. and Ehrlich, D., Anal. Chem. 2000, 72, 3129-3137.

92. Schmalzing, D., Belenky, A., Novotny, M. A., Koutny, L., Salas-Solano, O., El-Difrawy, S., Adourian, A., Matsudaira, P. and Ehrlich D. (2000) Microchip electrophoresis: a method for high-speed SNP detection. Nucleic Acids Res. 28, E43.

93. Schmalzing, D., Koutny, L., Salas-Solano, O., Adourian, A., Matsudaira, P., Ehrlich, D., Electrophoresis 1999, 20, 3066-3077.

94. Schwinefus, J. J. and Morris M. D. (1998) Band broadening during dc and field inversion capillary electrophoresis of □ DNA in dilute hydroxyethylcellulose solutions. Analyst 123, 1481-1485.

95. Schwinefus, J. J., Morris, M. D., Analyst. 1998, 123, 1481-1485.

96. Serwer P., Hayes, S. J., Moreno, E. T. and Park, C. Y. (1992) A small (58-nm) attached sphere perturbs the sieving of 40-80-kilobase DNA in 0.2-2.5% agarose gels: analysis of bacteriophage T7 capsid-DNA complexes by use of pulsed field electrophoresis. Biochemistry 31, 8397-8405.

97. Serwer, G. A. Griess: Anal. Chim. Acta 372,299 (1998).

98. Serwer, G. A. Griess: J. Chromatogr. B 722, 179 (1999).

99. Serwer, P. (1988) The source of energy for bacteriophage DNA packaging: An osmotic pump explains the data. Biopolymers 27, 165-169.

100. Serwer, P. (1990) Sieving by agarose gels and its use during pulsed-field electrophoresis. Biotechnol. Genet. Eng. Rev. 8, 319-343.

101. Serwer, P. and Hayes, S. J. (2001) Partially condensed DNA conformations observed by single molecule fluorescence microscopy. Biophys J., in press.

102. Serwer, P., Hayes, S. J., Moreno, E. T., Park, C. Y., Biochemistry 1992, 31, 8397-8405.

103. Serwer, S. J. Hayes, E. T. Moreno, C. Y. Park: Biochemistry 31, 8397 (1992).

104. Seto D., Seto J., Deshpande, P. and Hood, L. (1995) DMSO resolves certain compressions and signal dropouts in fluorescent dye labeled primer-based DNA sequencing reactions. DNA Sequence. 5, 131-140.

105. Shiraishi, M., Lerman, L. S., Sekiya T., Proc. Natl. Acad. Sci. USA 1995, 92, 4229-4233.

106. Slater, G. W. and Drouin, G., Electrophoresis 1992, 13, 574-582.

107. Slater, G. W., Desruisseaux, C., Hubert, S. J., Mercier, J. F., Labrie, J., Boileau, J., Tessier, F., Pepin, M. P., Electrophoresis 2000, 21, 3873-3887.

108. Slater, G. W., Desruisseaux, C., Hubert, S., J., Mercier, J. F., Labrie, J., Boileau,J., Tessier, F. and Pepin,M. P. (2000) Theory of DNA electrophoresis: a look at some current challenges. Electrophoresis 21, 3873-3887.

109. Slater, G. W., Mayer, P. and Grossman, P. D. (1995) Diffusion, Joule heating, and band broadening in capillary gel electrophoresis of DNA. Electrophoresis. 16, 75-83.

110. Slater, G. W., Mayer, P., Hubert S. J. and Drouin, G. (1994) The biased reptation model of DNA gel electrophoresis: a user guide for constant field mobilities. Appl. Theor. Electrophor. 4, 71-79.

111. Slater, G. W., Turmel, C., Lalande, M., Noolandi, J., Biopolymers 1989, 28, 1793-1799.

112. Smith, D. E., Tans, S. J., Smith, S. B., Grimes, S., Anderson, D. L. and Bustamante, C. (2001) The bacteriophage phi29 portal motor can package DNA against a large internal force. Nature 413, 748-752.

113. Song, J. M. and Yeung, E. S. (2001) Optimization of DNA electrophoretic behavior in poly(vinyl pyrrolidone) sieving matrix for DNA sequencing. Electrophoresis. 22, 748-754.

114. Song, J. M., Yeung, E. S., Electrophoresis 2001,22,748-754.

115. Song, L, Liang, D., Fang, D. and Chu, B. (2001a) Fast DNA sequencing up to 1000 bases by capillary electrophoresis using poly(N,N-dimethylacrylamide) as a separation medium. Elecropphoresis 22, 1987-1996.

116. Song, L., Liang, D., Chen, Z., Fang, D. and Chu, B. (2001b) DNA sequencing by capillary electrophoresis using mixtures of polyacrylamide and poly(N,N- dimethylacrylamide). J. of Chromatogr. 915, 231-239.

117. Staden, R., Judge, D. P. and Bonfield, J. K. (2001) Sequence assembly and finishing methods. Meth. Biochem. Anal. 43, 303-322.

118. Sudor, J., Barbier, V., Thirot, S., Godfrin, D., Hourdet D., Millequant, M., Blanchard, J. and Viovy, J. L. (2001) New block-copolymer thermoassociating matrices for DNA sequencing: effect of molecular structure on rheology and resolution. Electrophoresis. 22, 720-728.

119. Sun M., Son M. and Serwer, P. (1997) Formation and cleavage of a DNA network during in vitro bacteriophage T7 DNA packaging: light microscopy of DNA metabolism. Biochemistry. 36, 13018-13026.

120. Sun, M., Louie, D. and Serwer, P. (1999) Single-event analysis of the packaging of bacteriophage T7 DNA concatemers in vitro. Biophys. J. 77, 1627-1637.

121. Sunyaev, S., Lathe, W. 3rd and Bork, P. (2001) Integration of genome data and protein structures: prediction of protein folds, protein interactions and “molecular phenotypes” of single nucleotide polymorphisms. Curr. Opin. Struct. Biol. 11, 125-130.

122. T. N. Thornhill: J. Phys. D-Appl. Phys. 31, 253 (1998).

123. Timchenko, L. T. and Caskey, C. T. (1999) Triplet repeat disorders: discussion of molecular mechanisms. Cell. Mol. Life Sci. 55, 1432-1447.

124. Todd, R., Donoff, R. B., Kim, Y., Wong, D. T., J. Oral and Maxillofacial Surgery 2001, 59, 660-667.

125. Turmel, C., Brassard E and Noolandi, J. (1992) Manual sequencing using pulsed field. Electrophoresis 13, 620-622.

126. Turmel, C., Brassard, E, Noolandi, J., Electrophoresis 1992, 13, 620-622.

127. Ulanovsky L. (1992) Trapping electrophoresis of end-modified DNA in polyacrylamide gels. Meth. Enzymol. 216, 54-68.

128. Ulanovsky, G. Drouin, W. Gilbert: Nature 343, 190 (1990).

129. Ulanovsky, L., Drouin G., Gilbert, W., Nature 1990, 343, 190-192.

130. Vainer, M., Enad, S., Dolnik, V., Xu, D., Bashkin, J., Marsh, M., Tu O., Harris, D. W., Barker, D. L., Mansfield, E. S., Genomics 1997,41, 1-9.

131. Viovy, J. L. (1996) Reptation theories of electrophoresis. Mol. Biotechnol. 6, 31-46, 1996.

132. Viovy, J. L. (2000) Electrophoresis of DNA and other polyelectrolytes: Physical mechanisms. Rev. Mod. Phys. 72, 813-872.

133. Viovy, J.-L., Rev. Mod. Phys. 2000, 72, 813-872.

134. Voss, K. O., Roos, H. P. and Dovichi, N. J. (2001) The effect of temperature oscillations on DNA sequencing by capillary electrophoresis. Anal. Chem. 73, 1345-1349.

135. W. Slater, C. Desruisseaux, S. J. Hubert, J. -F. Mercier, J. Labrie, J. Boileau, F. Tessier, M. P. Pepin: Electrophoresis 21, 3873 (2000).

136. Walther, D., Bartha, G., Morris, M., Genome Res. 2001, 11, 875-888.

137. Washizu, M. and Kurosawa, O. (1990) Electrostatic manipulation of DNA in microfabricated structures. IEEE Trans. Industry Appl. 26, 1165-1172.

138. Wei, W. and Yeung, E. S. (2001) DNA capillary electrophoresis in entangled dynamic polymers of surfactant molecules. Anal. Chem. 73, 1776-1783.

139. Wei, W. and Yeung, E. S. (2000) improvements in DNA sequencing by capillary electrophoresis at elevated temperature using poly(ethylene oxide) as a sieving matrix. J. of Chromatogr. B, Biomed. Sci. and Appl. 745, 221-230.

140. Wei, W., Yeung, E. S., J. Chromatogr. B 2000, 745, 221-230, 2000.

141. Weinshenker, B. G., Hebrink, D. D., Gacy , A. M., McMurray, C. T., Biotechniques, 1998, 25, 68-72.

142. Wendl, M. C., Marra, M. A., Hillier, L. W., Chinwalla, A. T., Wilson, R. K. and Waterston, R. H. (2001) Theories and applications for sequencing randomly selected clones. Genome Res. 11, 274-280.

143. Witten, T. A. (1988) Associating polymers and shear thickening. J. Phys. (France) 49, 1055-1063.

144. -Y. Wang, T. Elston, A. Mogilner, G. Oster: Biophys. J. 74, 1186.

145. Yamakawa, H., Nakajima, D. and Ohara, O. (1996) Identification of sequence motifs causing band compressions on human CDNA sequencing. DNA Res. 3, 81-86.

146. Zhou, A. W. Miller, Z. Sosic, B. Buchholz, A. E. Barron, L. Kotler, B. L. Karger: Anal. Chem. 72, 1045 (2000).

147. Zhou, H., Miller, A. W., Sosic, Z., Buchholz, B., Barron, A. E., Kotler, L., Karger, B. L., Anal. Chem., 2000,72, 1045-1052.

148. Zhou, H., Miller, A. W., Sosic, Z., Buchholz, B., Barron, A. E., Kotler, L. and Karger, B. L., Anal. Chem. 2000,72, 1045-1052.

149. Zhou, H., Miller, A. W., Sosic, Z., Buchholz, B., Barron, A. E., Kotler, L. and Karger, B. L. (2000) DNA sequencing up to 1300 bases in two hours by capillary electrophoresis with mixed replaceable linear polyacrylamide solutions. Anal. Chem. 72, 1045-1052.

1-36. (canceled)

37. A method comprising:
(a) providing an analysis stage for analyzing DNA fragments where the analysis stage comprises a constant electric field for moving the DNA fragments in a first direction; and
(b) providing an enhancement stage for increasing the separation between peaks of the DNA fragments where the enhancement stage comprises a pulsed electrical field for moving the DNA fragments in a second direction and where the pulsed electrical field is generated by an electrophoretic ratchet.

38. The method of claim 37, the analysis stage comprising a first analysis stage and a second analysis stage.

39. The method of claim 38, further comprising re-analyzing the DNA fragments using the second analysis stage.

40. The method of claim 38, where the enhancement stage is between the first analysis stage and the second analysis stage.

41. The method of claim 37, the step of providing the analysis stage for analyzing DNA fragments further comprising analyzing the DNA fragments more than once for reducing errors.

42. The method of claim 37, further comprising stopping the first analysis stage.

43. The method of claim 37, the first direction comprising a forward direction and the second direction comprising a reverse direction.

44. The method of claim 37, further comprising repeating steps (a) and (b).

45. A method comprising:
(a) providing DNA fragments;
(b) applying a first constant electrical field for analyzing the DNA fragments;
(c) applying a pulsed electrical field for increasing separation between peaks of the DNA fragments, where the pulsed electrical field is generated by an electrophoretic ratchet; and
(d) applying a second constant electrical field for analyzing the DNA fragments.

46. The method of claim 45, where in steps (b) and (d), the DNA fragments are moved in a forward direction.

47. The method of claim 45, where in step (c), the DNA fragments are moved in a reverse direction.

48. The method of claim 45, further comprising repeating steps (b), (c) and (d).

49. A method comprising:
providing a DNA sequencing ladder;
applying a first cycle on the DNA sequencing ladder comprising:
a first analysis stage for analyzing a first DNA fragment of the DNA sequencing ladder, where a constant electrical field of the first analysis stage moves the first DNA fragment in a first direction; and
an enhancement stage for increasing separation between peaks of the DNA sequencing ladder, where the enhancement stage comprises a pulsed electrical field being generated by an electrophoretic ratchet for moving the DNA sequencing ladder in a second direction;
applying a second cycle on the DNA sequencing ladder, the second cycle comprising a second analysis stage for analyzing a second DNA fragment; and
repeating the first and second cycles.

50. The method of claim 49, where a portion of the second DNA fragment is analyzed in the first analysis stage.

51. The method of claim 49, where the second DNA fragment is longer than the first DNA fragment.

52. The method of claim 49, where the first direction comprises a forward direction and the second direction comprises a reverse direction.

53. The method of claim 49, further comprising stopping the first analysis stage prior to the enhancement stage.

54. The method of claim 49, where the step of applying the second cycle further comprises moving the second DNA fragment in the first direction.

55. Computer readable medium comprising machine readable instructions for:
providing a DNA sequencing ladder;
applying a first cycle on the DNA sequencing ladder comprising:
a first analysis stage for analyzing a first DNA fragment of the DNA sequencing ladder, where a constant electrical field of the first analysis stage moves the first DNA fragment in a first direction; and
an enhancement stage for increasing separation between peaks of the DNA sequencing ladder, where the enhancement stage comprises a pulsed electrical field being generated by an electrophoretic ratchet for moving the DNA sequencing ladder in a second direction;
applying a second cycle on the DNA sequencing ladder, the second cycle comprising a second analysis stage for analyzing a second DNA fragment; and
repeating the first and second cycles.
